(19) United States
(12) Patent Application Publication
(10) Pub. No.: US 2017/0349660 A1
(43) Pub. Date: Dec. 7, 2017

(54) BISPECIFIC ANTIBODIES THAT BIND CD123 AND CD3

(71) Applicant: Xencor. Inc., Monrovia, CA (US)

(72) Inventors: Michael Wayne Saville, San Diego, CA (US); Paul Foster, Rancho Santa Fe, CA (US)

(21) Appl. No.: 15/611,361

(22) Filed: Jun. 1, 2017

Related U.S. Application Data
(60) Provisional application No. 62/344,317, filed on Jun. 1, 2016.

(51) Int. Cl. C07K 16/28; A61K 39/395; A61K 45/06; C07K 16/30; A61K 39/00

(52) U.S. Cl. CPC C07K 16/2866; A61K 45/06; C07K 16/2809; C07K 16/3061; A61K 39/39558; A61K 2039/505; A61K 2039/54; C07K 2317/31; C07K 2317/92; A61K 2039/545

(57) ABSTRACT

The present invention is directed to novel bispecific anti-CD123 x anti-CD3 antibodies.

### PRIORITY CLAIM

This application claims priority to U.S. Provisional Application Serial No. 62/344,317, filed Jun. 1, 2016 which is expressly incorporated by reference in its entirety.

The instant application contains a Sequence Listing which has been submitted electronically in ASCII format and is hereby incorporated by reference in its entirety. Said ASCII copy, created on May 30, 2017, is named 067461-5192-US_ST25.txt and is 45,294 bytes in size.

### BACKGROUND OF THE INVENTION

Antibody-based therapeutics have been used successfully to treat a variety of diseases, including cancer and autoimmune/inflammatory disorders. Yet improvements to this class of drugs are still needed, particularly with respect to enhancing their clinical efficacy. One avenue being explored is the engineering of additional and novel antigen binding sites into antibody-based drugs such that a single immunoglobulin molecule co-engages two different antigens.. Because the considerable diversity of the antibody variable region (Fv) makes it possible to produce an Fv that recognizes virtually any molecule, the typical approach to the generation of such bispecific antibodies is the introduction of new variable regions into the antibody.

A number of alternate antibody formats have been explored for bispecific targeting (Chames & Baty, 2009, mAbs 1[6]:1-9; Holliger & Hudson, 2005, Nature Biotechnology 23[9]:1126-1136; Kontermann, mAbs 4(2):182 (2012), all of which are expressly incorporated herein by reference). Initially, bispecific antibodies were made by fusing two cell lines that each produced a single monoclonal antibody (Milstein et al., 1983, Nature 305:537-540). Although the resulting hybrid hybridoma or quadroma did produce bispecific antibodies, they were only a minor population, and extensive purification was required to isolate the desired antibody. An engineering solution to this was the use of antibody fragments to make bispecifics. Because such fragments lack the complex quaternary structure of a full length antibody, variable light and heavy chains can be linked in single genetic constructs. Antibody fragments of many different forms have been generated, including diabodies, single chain diabodies, tandem scFvs, and Fab₂ bispecifics (Chames & Baty, 2009, mAbs 1[6]:1-9; Holliger & Hudson, 2005, Nature Biotechnology 23[9]:1126-1136; expressly incorporated herein by reference). While these formats can be expressed at high levels in bacteria and may have favorable penetration benefits due to their small size, they clear rapidly in vivo and can present manufacturing obstacles related to their production and stability. A principal cause of these drawbacks is that antibody fragments typically lack the constant region of the antibody with its associated functional properties, including larger size, high stability, and binding to various Fc receptors and ligands that maintain long half-life in serum (i.e. the neonatal Fc receptor FcRn) or serve as binding sites for purification (i.e. protein A and protein G).

More recent work has attempted to address the shortcomings of fragment-based bispecifics by engineering dual binding into full length antibody -like formats (Wu et al., 2007, Nature Biotechnology 25[11]:1290-1297; U.S. Ser. Np. 12/477,711; Michaelson et al., 2009, mAbs 1[2]:128-141; PCT/US2008/074693; Zuo et al., 2000, Protein Engineering 13[5]:361-367; U.S. Ser. No. 9/865,198; Shen et al., 2006, J Biol Chem 281[16]:10706-10714; Lu et al., 2005, J Biol Chem 280[20]:19665-19672; PCT/US2005/025472; expressly incorporated herein by reference). These formats overcome some of the obstacles of the antibody fragment bispecifics, principally because they contain an Fc region. One significant drawback of these formats is that, because they build new antigen binding sites on top of the homodimeric constant chains, binding to the new antigen is always bivalent.

For many antigens that are attractive as co-targets in a therapeutic bispecific format, the desired binding is monovalent rather than bivalent. For many immune receptors, cellular activation is accomplished by cross-linking of a monovalent binding interaction. The mechanism of cross-linking is typically mediated by antibody/antigen immune complexes, or via effector cell to target cell engagement. For example, the low affinity Fc gamma receptors (FcγRs) such as FcγRIIa, FcγRIIb, and FcγRIIIa bind monovalently to the antibody Fc region. Monovalent binding does not activate cells expressing these FcγRs; however, upon immune complexation or cell-to-cell contact, receptors are cross-linked and clustered on the cell surface, leading to activation. For receptors responsible for mediating cellular killing, for example FcγRIIIa on natural killer (NK) cells, receptor cross-linking and cellular activation occurs when the effector cell engages the target cell in a highly avid format (Bowles & Weiner, 2005, J Immunol Methods 304:88-99, expressly incorporated by reference). Similarly, on B cells the inhibitory receptor FcγRllb downregulates B cell activation only when it engages into an immune complex with the cell surface B-cell receptor (BCR), a mechanism that is mediated by immune complexation of soluble IgG's with the same antigen that is recognized by the BCR (Heyman 2003, Immunol Lett 88[2]:157-161; Smith and Clatworthy, 2010, Nature Reviews Immunology 10:328-343; expressly incorporated by reference). As another example, CD3 activation of T-cells occurs only when its associated T-cell receptor (TCR) engages antigen-loaded MHC on antigen presenting cells in a highly avid cell-to-cell synapse (Kuhns et al., 2006, Immunity 24:133-139). Indeed nonspecific bivalent cross-linking of CD3 using an anti-CD3 antibody elicits a cytokine storm and toxicity (Perruche et al., 2009, J Immunol 183[2]:953-61; Chatenoud & Bluestone, 2007, Nature Reviews Immunology 7:622-632; expressly incorporated by reference). Thus for practical clinical use, the preferred mode of CD3 co-engagement for redirected killing of targets cells is monovalent binding that results in activation only upon engagement with the co-engaged target.

CD123, also known as interleukin-3 receptor alpha (IL-3Rα), is expressed on dendritic cells, monocytes, eosinophils and basophils. CD123 is also constitutively expressed by committed hematopoietic stem/progenitor cells, by most of the myeloid lineage (CD13+, CD14+, CD33+, CD15low), and by some CD19+ cells. It is absent from CD3+ cells.

Accordingly, there is a need for improved bispecific anti-CD-123 x anti-CD3 antibodies and the use of such antibodies for use in therapy.

### BRIEF SUMMARY OF THE INVENTION

In one aspect, the present invention provides a method for treating a CD123-expressing cancer, e.g., a hematologic cancer, e.g., leukemia, in a human subject, comprising: administering to the human subject having a CD123-expressing cancer, e.g., a hematologic cancer, e.g., leukemia, an intravenous dose of between about 1 ng/kg and about 800 ng/kg of a bispecific anti-CD123 x anti-CD3 antibody (e.g., XENP14045) once every 6-8 days for a time period sufficient to treat the CD123-expressing cancer, e.g., the hematologic cancer, e.g., leukemia.

In one aspect, provided herein is a bispecific anti-CD123 x anti-CD3 antibody (e.g., XENP14045) for use in treating a CD123-expressing cancer, e.g., a hematologic cancer, e.g., leukemia, in a human subject having a CD123-expressing cancer by administering to the human subject between about 1 ng/kg and about 800 ng/kg of an intravenous dose of the bispecific anti-CD123 x anti-CD3 antibody (e.g., XENP14045) once every 6-8 days for a time period sufficient to treat the CD123-expressing cancer, e.g., the hematologic cancer, e.g., leukemia.

In one aspect, the present invention provides a method for treating a CD123-expressing cancer, e.g., a hematologic cancer, e.g., leukemia, in a human subject, comprising: administering to the human subject having a CD123-expressing cancer, e.g., a hematologic cancer, e.g., leukemia, an intravenous dose of between about 75 ng/kg and about 750 ng/kg of a bispecific anti-CD123 x anti-CD3 antibody (e.g., XENP14045) monthly for a time period sufficient to treat the CD123-expressing cancer, e.g., the hematologic cancer, e.g., leukemia.

In one aspect, provided herein is a bispecific anti-CD123 x anti-CD3 antibody (e.g., XENP14045) for use in treating a CD123-expressing cancer, e.g., a hematologic cancer, e.g., leukemia, in a human subject having a CD123-expressing cancer by administering to the human subject between about 75 ng/kg and about 750 ng/kg of an intravenous dose of the bispecific anti-CD123 x anti-CD3 antibody (e.g., XENP14045) montly for a time period sufficient to treat the CD123-expressing cancer, e.g., the hematologic cancer, e.g., leukemia.

In one aspect, the present invention provides a method for treating a CD123-expressing cancer, e.g., a hematologic cancer, e.g., leukemia, in a human subject, comprising: administering to the human subject having a CD123-expressing cancer, e.g., a hematologic cancer, e.g., leukemia, an intravenous dose of between about 75 ng/kg and about 750 ng/kg of a bispecific anti-CD123 x anti-CD3 antibody (e.g., XENP14045) every other week for a time period sufficient to treat the CD123-expressing cancer, e.g., the hematologic cancer, e.g., leukemia.

In one aspect, provided herein is a bispecific anti-CD123 x anti-CD3 antibody (e.g., XENP14045) for use in treating a CD123-expressing cancer, e.g., a hematologic cancer, e.g., leukemia, in a human subject having a CD123-expressing cancer by administering between about 75 ng/kg and about 750 ng/kg of an intravenous dose of the anti-CD123 x anti-CD3 antibody (e.g., XENP14045) every other week for a time period sufficient to treat the CD123-expressing cancer, e.g., the hematologic cancer, e.g., leukemia.

In some embodiments, the intravenous dose is: between about 2 ng/kg and about 4 ng/kg; or between about 9 ng/kg and about 11 ng/kg; or between about 25 ng/kg and about 35 ng/kg; or between about 70 ng/kg and about 80 ng/kg; or between about 125 ng/kg and about 175 ng/kg; or between about 275 ng/kg and about 325 ng/kg; or between about 475 ng/kg and about 525 ng/kg; or between about 725 ng/kg and about 775 ng/kg.

In one aspect, the intravenous dose according to the present invention is administered to a human subject between about 1 hour and about 3 hours. In some embodiments, the time period sufficient to treat a CD123-expressing cancer, e.g., a hematologic cancer, e.g., leukemia in a human subject is between about 3 weeks and 9 weeks. In some embodiments, the time period sufficient to treat a CD123-expressing cancer, e.g., a hematologic cancer, e.g., leukemia in a human subject is between about 4 weeks and 9 weeks.

In one aspect, the bispecific anti-CD123 x anti-CD3 antibody according to the present invention is XENP14045 as described herein. In such embodiments, the XENP14045 bispecific anti-CD123 x anti-CD3 antibody includes a first monomer comprising SEQ ID NO: 1, a second monomer comprising SEQ ID NO: 2, and a light chain comprising SEQ ID NO: 3.

In one aspect, a human subject that is being treated according to the present invention has leukemia, for example, leukemia selected from the group consisting of acute myeloid leukemia (AML), chronic myeloid leukemia (CML), acute lymphocytic leukemia (ALL), blastic plasmacytoid dendritic cell neoplasm, and hairy cell leukemia (HCL). In some embodiments, leukemia is acute myeloid leukemia (AML). In some embodiments, AML is blastic plasmacytoid dendritic cell neoplasm (BPDCN). In some embodiments, leukemia is ALL. In some embodiments, ALL is B-cell acute lymphocytic leukemia (B-ALL).

In one aspect, the methods and antibodies of the present invention further comprise, prior to the administering, assessing the weight of the human subject.

In one aspect, the methods and antibodies of the present invention further comprise administering to said subject one or more other therapies. In some embodiments, said other therapies include a chemotherapy, for example, a chemotherapy selected from the group consisting of: anthracycline (e.g., idarubicin, daunorubicin, doxorubicin (e.g., liposomal doxorubicin)), a anthracenedione derivative (e.g., mitoxantrone), a vinca alkaloid (e.g., vinblastine, vincristine, vindesine, vinorelbine), an alkylating agent (e.g., cyclophosphamide, deacarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, cytarabine, pyrimidine analogs, purine analogs and adenosine deaminase inhibitors (e.g., fludarabine)), an mTOR inhibitor, a proteasome inhibitor (e.g., aclacinomycin A, gliotoxin or bortezomib), an immunomodulator such as thalidomide or a thalidomide derivative (e.g., lenalidomide). In some embodiments, said other therapies include a therapy that ameliorate side effects, for example, a therapy that is selected from the group consisting of: steroid (e.g., corticosteroid, e.g., methylprednisolone, hydrocortisone), an inhibitor of TNFα, inhibitor of IL-1R, and an inhibitor of IL-6. In some embodiments, said other therapies are a combination of a corticosterioid (e.g., methylprednisolone, hydrocortisone) and Benadryl and Tylenol, wherein said corticosterioid, Benadryl and Tylenol are administered to the subject prior to the administration of the anti-CD123 x anti-CD3 antibody (e.g., XENP14045).

### BRIEF DESCRIPTION OF THE DRAWINGS

FIG. 1 depicts a particularly useful bispecific format of the invention, referred to as a “bottle opener”, which is also the format of XENP14045. It should be noted that the scFv and Fab domains can be switched (e.g. anti-CD3 as a Fab, and anti-CD123 as a scFv).

FIG. 2 depicts the sequences of the three polypeptide chains that make up XENP14045, an anti-CD123 x anti-CD3 antibody of particular use in the present invention. The CDRs are underlined and the junction between domains is denoted by a slash (“/”). The charged scFv linker is double underlined; as will be appreciated by those in the art, the linker may be substituted with other linkers, and particularly other charged linkers that are depicted in FIG. 7 of US Publication Number 2014/0288275, or other non-charged linkers (SEQ ID NO:441 of US Publication Number 2014/0288275).

FIG. 3 depicts the engineering of a number of anti-CD123 Fab constructs to increase affinity to human CD123 and stability of the 7G3 H1L1 construct, including the amino acid changes.

FIG. 4 depicts the properties of final affinity and stability optimized humanized variants of the parental 7G3 murine antibody.

FIG. 5A-5B depicts additional anti-CD123 Fab sequences of the invention, with the CDRs underlined.

FIG. 6 depicts additional anti-CD123 x anti CD3 sequences of the invention. The CDRs are underlined and the junction between domains is denoted by a slash (“/”). The charged scFv linker is double underlined; as will be appreciated by those in the art, the linker may be substituted with other linkers, and particularly other charged linkers that are depicted in FIG. 7 of US Publication Number 2014/0288275, or other non-charged linkers (SEQ ID NO:441 of US Publication Number 2014/0288275).

FIG. 7A-7D depicts additional bispecific formats of use in the present invention, as are generally described in FIG. 1 and the accompanying Legend and supporting text of U.S. Ser. No. 14/952,714 (incorporated herein by reference).

FIG. 8 depicts RTCC with intact or T cell depleted PBMC against KG-1a target cells. Effector cells (400k), intact or magnetically-depleted PBMC were incubated with carboxyfluorescein succinimidyl ester-labeled KG-1a target cells (10k) for 24 hours and stained with annexin V for cell death.

FIG. 9 depicts CD123hiCD33hi depletion over a dose range of XmAb14045 in AML patient PBMC. Five AML patient PBMC samples were incubated with a dose range of XmAb14045 (0.12 to 90 ng/mL) for 6 days, and live cells were gated to count CD123hiCD33hi target cells. The lowest concentration (0.04 ng/mL) point is the no drug control for plotting on logarithmic scale. Each point is normalized to account for cell count variability.

FIG. 10 depicts Ki67 levels in T cells from AML patient PBMC with XmAb14045. Five AML patient PBMC samples were incubated with a dose range of XmAb14045 (0.12 to 90 ng/mL) for 6 days, and live cells were gated for CD4+ and CD8+ T cells to count Ki67+ cells. The lowest concentration (0.04 ng/mL) point is the no drug control, for plotting on a logarithmic scale.

FIG. 11 depicts number of AML blasts in patient PBMCs treated with XmAb14045. PBMC from a single AML patient was incubated with 9 or 90 ng/mL XmAb14045 for 24 or 48 hours and blast counts were plotted. Normal donor PBMCs were also used as a control.

FIG. 12 depicts leukemic blast cells in AML patient PBMC. PBMCs from six AML patients were incubated with antibodies for 48 hours and blasts were counted and plotted. One donor (AML #1) did not have XENP13245 treatment and each line is a single donor.

FIG. 13 depicts KG-1a tumor cell apoptosis with AML PBMC. Carboxyfluorescein succinimidyl ester-labeled CD123+ KG-1a cells were added to the PBMC to examine target cell cytotoxicity stimulated by the AML effector T cells. Staining with the apoptosis marker annexin-V was used to detect KG-1a cell death after 48 hours of incubation.

FIG. 14 depicts effect of XmAb14045 on tumor burden over time in a mouse xenograft model of AML.

FIG. 15 depicts reduction of tumor burden after 3 weekly doses of XmAb14045.

FIG. 16 depicts effect of XmAb14045 on T cell number in a mouse xenograft model of AML. Peripheral blood CD45+CD8+ events by flow cytometry. Samples taken on Day 11 and 20 after XmAb14045 administration.

### DETAILED DESCRIPTION OF THE INVENTION

### I. Definitions

In order that the application may be more completely understood, several definitions are set forth below. Such definitions are meant to encompass grammatical equivalents.

By “CD3” or “cluster of differentiation 3” herein is meant a T-cell co-receptor that helps in activation of both cytooxic T-cell (e.g., CD8+ naïve T cells) and T helper cells (e..g, CD4+ naïve T cells) and is composed of four distinct chains: one CD3γ chain (e.g., Genbank Accession Numbers NM_000073 and MP_000064 (human)), one CD3δ chain (e.g., Genbank Accession Numbers NM_000732, NM_001040651, NP_00732 and NP_001035741 (human)), and two CD3ε chains (e.g., Genbank Accession Numbers NM_000733 and NP_00724 (human)). The chains of CD3 are highly related cell-surface proteins of the immunoglobulin superfamily containing a single extracellular immunoglobulin domain. The CD3 molecule associates with the T-cell receptor (TCR) and ζ-chain to form the T-cell receptor (TCR) complex, which functions in generating activation signals in T lymphocytes.

By “CD123” or “Cluster of Differentiation 123”or “CD123 antigen” or “interleukin-3 receptor alpha” or “IL3RA” or “interleukin3 receptor subunit alpha” is meant athe interleukin 3 specific subunit of a type I heterodimeric cytokine receptor (e.g., Genbank Accession Numbers NM_001267713, NM_002183, NP_001254642 and NP_002174 (human)). CD123 interacts with a signal transducing beta subunit to form interleukin-3 receptor, which helps in the transmission of interleukin 3. CD123 is found on pluripotent progenitor cells and induces tyrosine phosphorylation within the cell and promotes proliferation and differentiation within the hematopoietic cell lines. CD123 is expressed across acute myeloid leukemia (AML substypes, including leukemic stem cells

By “bispecific” or “bispecific anitbody” herein is meant any non-native or alternate antibody formats, including those described herein, that engage two different antigens (e.g., CD3 x CD123 bispecific antibodies).

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233# or E233( )designates a deletion of glutamic acid at position 233. Additionally, EDA233- or EDA233# designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. For example, homo-phenylalanine, citrulline, ornithine and noreleucine are considered synthetic amino acids for the purposes of the invention, and both D- and L-(R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CHL VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

By “amino acid” and “amino acid identity” as used herein is meant one of the 20 naturally occurring amino acids that are coded for by DNA and RNA.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcqammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and

FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cyl (Cyl) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

By “heavy constant region” herein is meant the CH1-hinge-CH2-CH3 portion of an antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. The two target antigens of the present invention are human CD3 and human CD123.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g. making the pI higher) then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “target cell” as used herein is meant a cell that expresses a target antigen.

By “variable region” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10-4 M, at least about 10-5 M, at least about 10-6 M, at least about 10-7 M, at least about 10-8 M, at least about 10-9 M, alternatively at least about 10-10 M, at least about 10-11 M, at least about 10-12 M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000-or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000-or more times greater for the epitope relative to a control, where KA or Ka refers to an association rate of a particular antibody-antigen interaction. Binding affinity is generally measured using a Biacore assay.

As used herein, the term “target activity” refers to a biological activity capable of being modulated by a selective modulator. Certain exemplary target activities include, but are not limited to, binding affinity, signal transduction, enzymatic activity, tumor growth, effects on particular biomarkers related to CD123 disorder pathology.

By “refractory” in the context of a cancer is intended the particular cancer is resistant to, or non-responsive to, therapy with a particular therapeutic agent. A cancer can be refractory to therapy with a particular therapeutic agent either from the onset of treatment with the particular therapeutic agent (i.e., non-responsive to initial exposure to the therapeutic agent), or as a result of developing resistance to the therapeutic agent, either over the course of a first treatment period with the therapeutic agent or during a subsequent treatment period with the therapeutic agent.

As used herein, the IC₅₀ refers to an amount, concentration or dosage of a particular test compound that achieves a 50% inhibition of a maximal response, such as inhibition of the biological activity of CD123, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

### II. Overview

The invention provides methods of treating a cancer that include cells expressing CD123 (“CD123-expressing cancer”), for example, a hematologic cancer, such as leukemia, through the administration of certain bispecific anti-CD123 x anti-CD3 antibodies at particular dosages. These particular dosages are reduced over those known in the art. The present invention also provides methods of combination therapies, for example, methods of treating a cancer that include cells expressing CD123 (“CD123-expressing cancer”), e.g., a hematologic cancer, such as leukemia, through the administration of certain bispecific anti-CD123 x anti-CD3 antibodies (e.g., XENP14045) in combination with one or more chemotherapies or therapies that can ameliorate side effects of an anti-CD123 x anti-CD3 antibody.

### III. Antibodies

The present invention is directed to the administration of bispecific anti-CD123 x anti-CD3 antibodies for the treatment of particular leukemias as outlined herein, as outlined in U.S. Ser. Nos. 14/952,714, 15/141,350, and 62/085,027, all of which are expressly incorporated herein by reference, particularly for the bispecific formats of the figures, as well as all sequences, Figures and accompanying Legends therein.

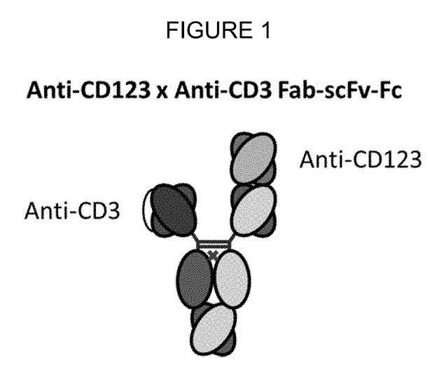

In some embodiments, the bispecific anti-CD123 x anti-CD3 antibodies have a “bottle opener” format as is generally depicted in FIG. 1. In this embodiment, the anti-CD3 antigen binding domain is the scFv-Fc domain monomer and the anti-CD123 antigen binding domain is the Fab monomer (terms as used in US Publication Nos. 2014/0288275 and 2014-0294823 as well as in U.S. Ser. No. 15/141,350, all of which are expressly incorporated by reference in their entirety and specifically for all the definitions, sequences of anti-CD3 antigen binding domains and sequences of anti-CD123 antigen binding domains).

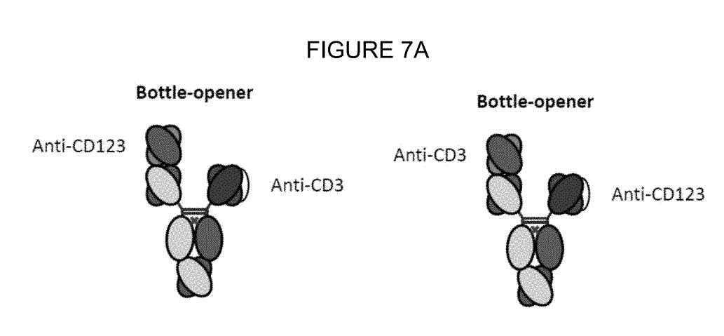

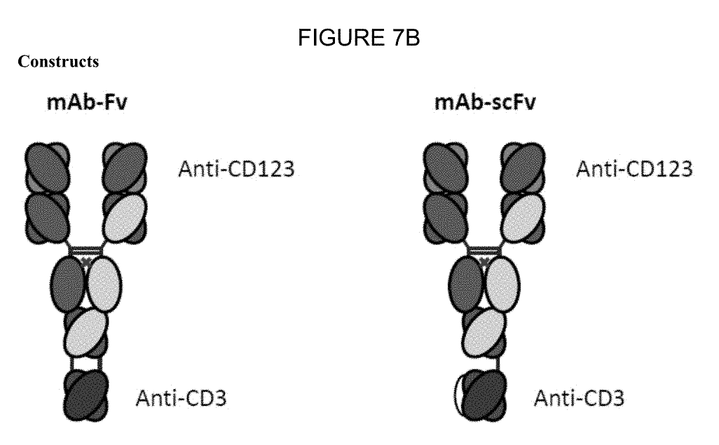

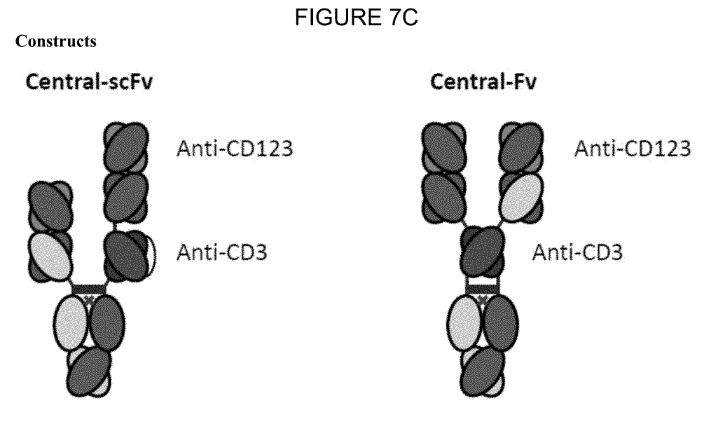

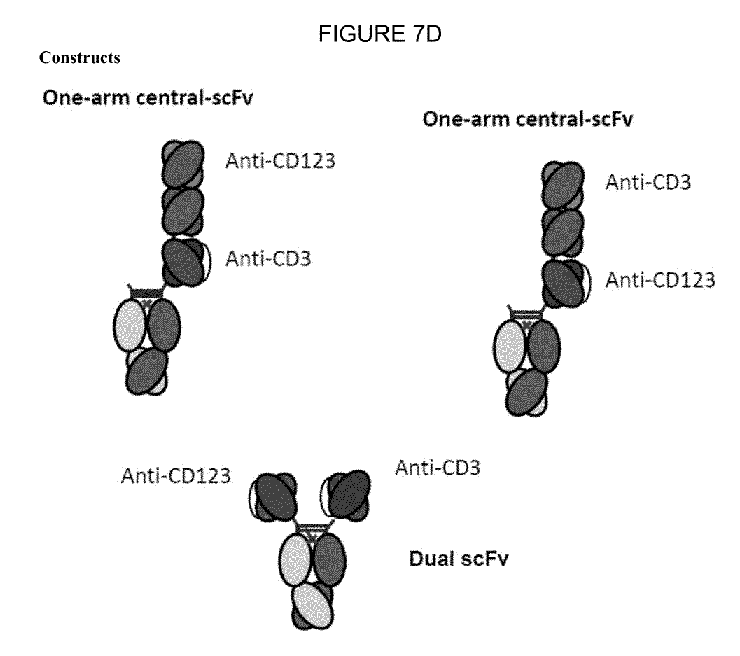

Alternate formats for the bispecific, heterodimeric anti-CD123 x anti-CD3 antibodies of the invention are shown in FIG. 7, which also generally rely on the use of Fabs and scFv domains in different formats.

In addition, it is also possible to make non-heterodimeric anti-CD123 x anti-CD3 bispecific antibodies as are known in the art, that can be dosed at the same dosage levels as described herein for the heterodimeric bispecific anti-CD123 x anti-CD3 antibodies.

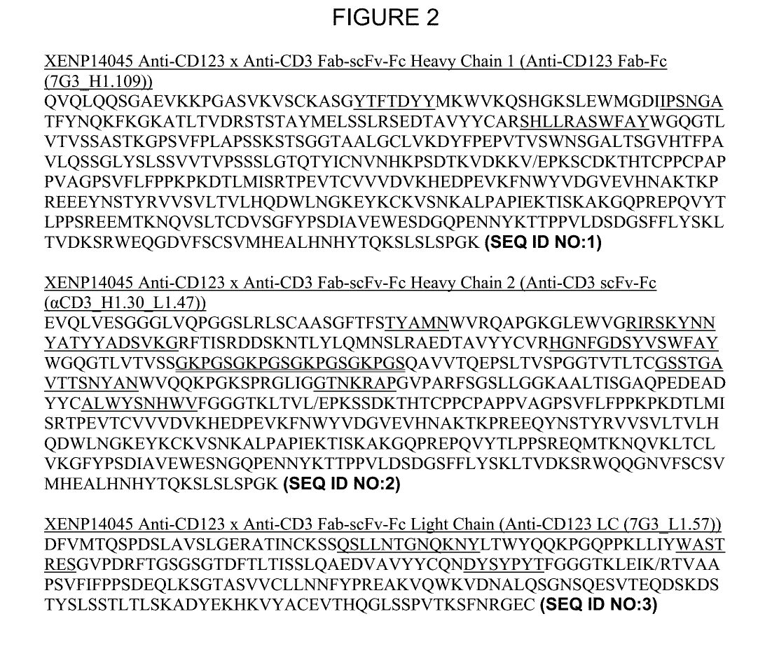

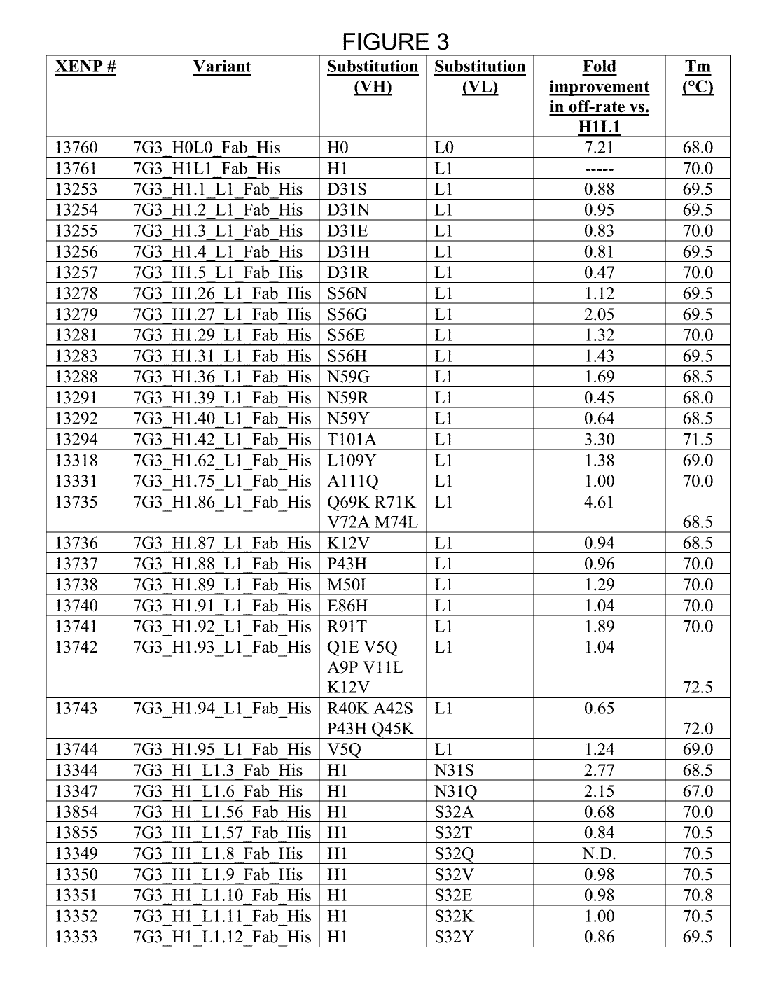

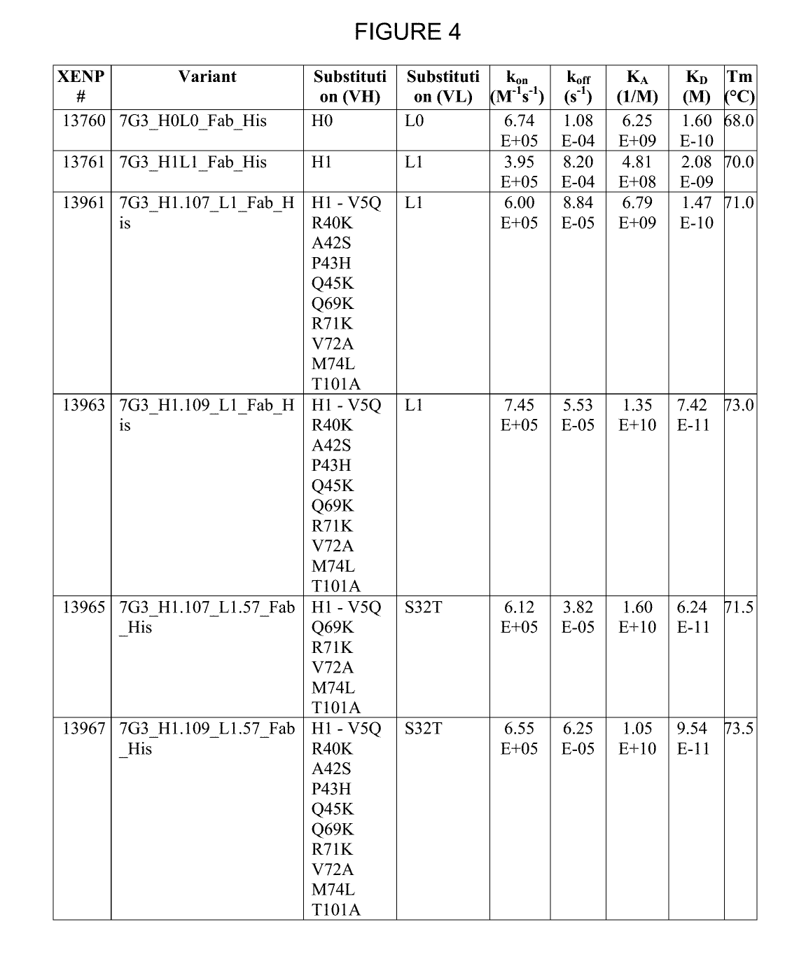

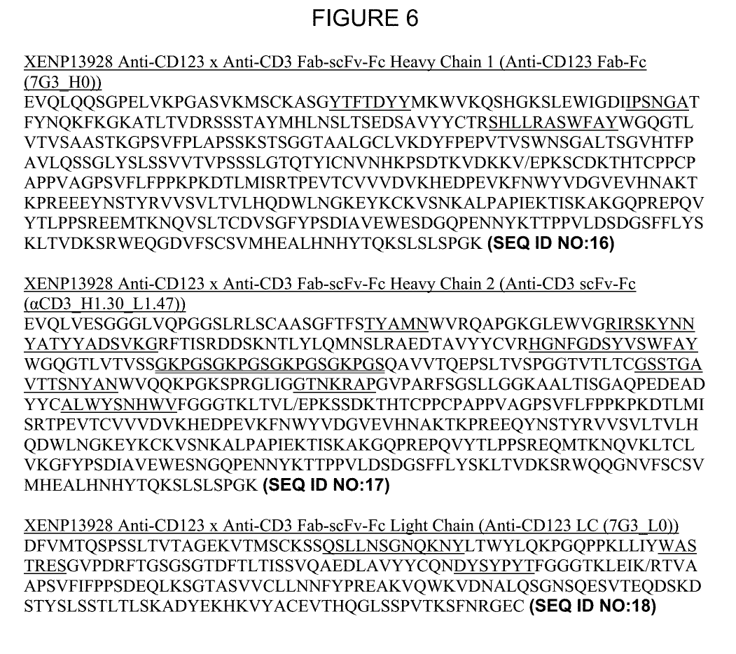

The anti-CD3 scFv antigen binding domain can have the sequence depicted in FIG. 2, or can be selected from:
1) the set of 6 CDRs (vhCDR1, vhCDR2, vhCDR3, vlCDR1, vlCDR2 and vlCDR3) from any anti-CD3 antigen binding domain sequence depicted in FIGS. 2 and 6 of US Publication No. 2014/0288275;
2) the variable heavy and variable light chains from any anti-CD3 antigen binding domain sequence depicted in FIGS. 2 and 6 of US Publication No. 2014/0288275;
3) the scFv domains from any anti-CD3 scFV sequence depicted in FIG. 2 of US Publication No. 2014/0288275;
4) other anti-CD3 variable heavy and variable light chains as are known in the art, that can be combined to form scFvs (or Fabs, when the format is reversed or an alternative format is used); and
5) any of the anti-CD3 antigen binding domains of FIGS. 2, 3, 4, 5, 6, and 7 of U.S. Ser. No. 14/952,714.

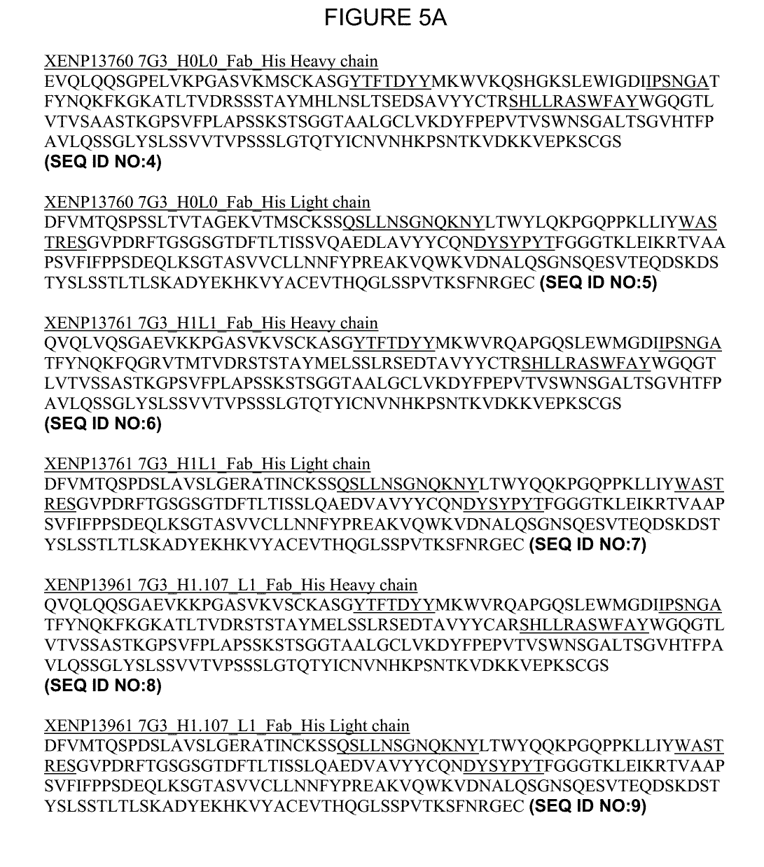

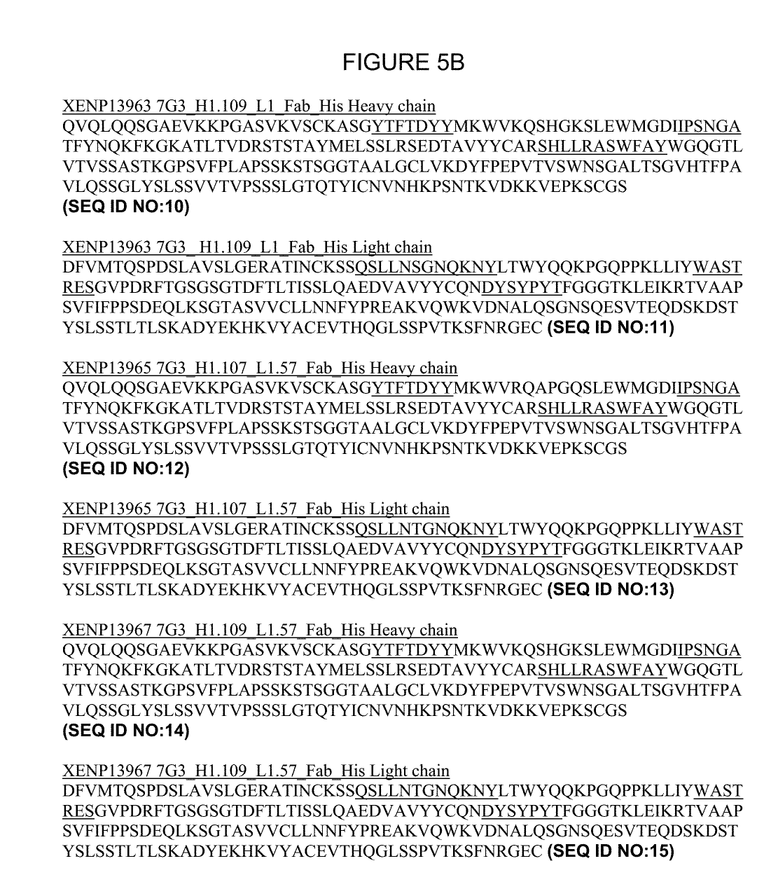

The anti-CD123 Fab binding domain can have the sequence depicted in FIG. 2 or 5, or can be selected from:
1) The set of 6 CDRs (vhCDR1, vhCDR2, vhCDR3, vlCDR1, vlCDR2 and vlCDR3) from any anti-CD123 antigen binding domain sequence depicted in USSN 62/085,027, including those depicted in FIGS. 2, 3 and 12;
2) The variable heavy and variable light chains from any anti-CD123 antigen binding domain sequence depicted in USSN 62/085,027, including those depicted in FIGS. 2, 3 and 12; and
3) Other anti-CD123 variable heavy and variable light chains as are known in the art, that can be combined to form Fabs (or scFvs, when the format is reversed or an alternative format is used).

One bispecific antibody of particular use in the present invention, XENP14045, is shown in FIG. 2. The XENP14045 bispecific antibody includes a first monomer comprising SEQ ID NO: 1, a second monomer comprising SEQ ID NO: 2, and a light chain comprising SEQ ID NO: 3.

The bispecific anti-CD123 x anti-CD3 antibodies of the invention are made as is known in the art. The invention further provides nucleic acid compositions encoding the bispecific anti-CD123 x anti-CD3 antibodies of the invention. As will be appreciated by those in the art, the nucleic acid compositions will depend on the format and scaffold of the bispecific anti-CD123 x anti-CD3 antibodies. Thus, for example, when the format requires three amino acid sequences, such as for the triple F format (e.g. a first amino acid monomer comprising an Fc domain and a scFv, a second amino acid monomer comprising a heavy chain and a light chain), three nucleic acid sequences can be incorporated into one or more expression vectors for expression. Similarly, some formats (e.g. dual scFv formats such as disclosed in FIG. 7) only two nucleic acids are needed; again, they can be put into one or two expression vectors.

As is known in the art, the nucleic acids encoding the components of the invention can be incorporated into expression vectors as is known in the art, and depending on the host cells used to produce the bispecific anti-CD123 x anti-CD3 antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments.

In some embodiments, nucleic acids encoding each monomer and the optional nucleic acid encoding a light chain, as applicable depending on the format, are each contained within a single expression vector, generally under different or the same promoter controls. In embodiments of particular use in the present invention, each of these two or three nucleic acids are contained on a different expression vector.

The heterodimeric bispecific anti-CD123 x anti-CD3 antibodies of the invention are made by culturing host cells comprising the expression vector(s) as is well known in the art. Once produced, traditional antibody purification steps are done, including an ion exchange chromatography step. As discussed in U.S. Ser. No. 14/205,248 and WO2014/145806, hereby incorporated by reference in their entirety and particularly for the discussions concerning purification, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI ) of each monomer so that such that each monomer has a different pI and the heterodimer also has a distinct pI, thus facilitating isoelectric purification of the “triple F” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

Once made, the bispecific anti-CD123 x anti-CD3 antibodies are administered to patients in dosages as outlined herein.

### III. a) Pharmaceutical Compositions and Pharmaceutical Administration

The bispecific anti-CD123 x anti-CD3 antibodies (e.g., XENP14045) of the invention can be incorporated into pharmaceutical compositions suitable for administration to a subject for the methods described herein, e.g., weekly, intravenous dosing. Typically, the pharmaceutical composition comprises a bispecific anti-CD123 x anti-CD3 antibody of the invention (e.g., XENP14045) and a pharmaceutically acceptable carrier. As used herein, “pharmaceutically acceptable carrier” includes any and all solvents, dispersion media, coatings, isotonic and absorption delaying agents, and the like that are physiologically compatible and are suitable for administration to a subject for the methods described herein. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as surfactants (such as nonionic surfactants) wetting or emulsifying agents, preservatives or buffers (such as an organic acid, which as a citrate), which enhance the shelf life or effectiveness of the bispecific anti-CD123 x anti-CD3 antibody (e.g., XENP14045). An example of pharmaceutically acceptable carriers include polysorbates (polysorbate-80). In an exemplary embodiment, the pharmaceutical composition comprises an antibody described herein, and a citrate. In an exemplary embodiment, the pharmaceutical composition comprises an antibody described herein, and a polysorbate. In an exemplary embodiment, the pharmaceutical composition comprises an antibody described herein, and a citrate and a polysorbate. In an exemplary embodiment, the pharmaceutical composition comprises an antibody described herein, and sodium citrate. In an exemplary embodiment, the pharmaceutical composition comprises an antibody described herein, and polysorbate-80. In an exemplary embodiment, the pharmaceutical composition comprises an antibody described herein, and sodium citrate and polysorbate-80. In an exemplary embodiment, the pharmaceutical composition comprises an antibody described herein, and sodium chloride. In an exemplary embodiment, the pharmaceutical composition comprises an antibody described herein, and sodium chloride and polysorbate-80. In an exemplary embodiment, the pharmaceutical composition comprises an antibody described herein, and sodium citrate and sodium chloride. In an exemplary embodiment, the pharmaceutical composition comprises an antibody described herein, and sodium citrate, sodium chloride, and polysorbate-80.

The pharmaceutical compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application. Exemplary compositions are in the form of injectable or infusible solutions, such as compositions similar to those used for passive immunization of humans with other antibodies. In an exemplary embodiment, the mode of administration is intravenous. In an exemplary embodiment, the antibody is administered by intravenous infusion or injection.

Pharmaceutical compositions typically must be sterile and stable under the conditions of manufacture and storage. The pharmaceutical composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated herein, as required, followed by filtered sterilization. Generally, dispersions are prepared by incorporating the antibody into a sterile vehicle that contains a basic dispersion medium and the required other ingredients from those enumerated herein. In the case of sterile powders for the preparation of sterile injectable solutions, in an exemplary embodiment, the method of preparation is vacuum drying and freeze-drying that yields a powder of the antibody plus any additional desired carrier from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The bispecific anti-CD123 x anti-CD3 antibodies of the present invention can be administered by a variety of methods known in the art. In an exemplary embodiment, the route/mode of administration is intravenous injection. As will be appreciated by the skilled artisan, the route and/or mode of administration will vary depending upon the desired results. In certain embodiments, the bispecific anti-CD123 x anti-CD3 antibody (e.g., XENP14045) may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyethylene glycol (PEG), polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

### IV. Methods of Treating Leukemia

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive build up of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

In an exemplary embodiment, the leukemia is selected from the group consisting of acute lymphocytic leukemia (ALL), acute myeloid leukemia (AML), chronic myeloid leukemia (CML), and hairy cell leukemia (HCL). In an exemplary embodiment, the leukemia is acute lymphocytic leukemia (ALL). In an exemplary embodiment, the leukemia is acute myeloid leukemia (AML). In an exemplary embodiment, the leukemia is chronic myeloid leukemia (CML). In an exemplary embodiment, the leukemia is chronic phase chronic myeloid leukemia. In an exemplary embodiment, the leukemia is accelerated phase chronic myeloid leukemia. In an exemplary embodiment, the leukemia is blast phase chronic myeloid leukemia. In an exemplary embodiment, the leukemia is hairy cell leukemia (HCL). In an exemplary embodiment, the leukemia is classic hairy cell leukemia (HCLc). In an exemplary embodiment, the leukemia is variant hairy cell leukemia (HCLv). In an exemplary embodiment, the leukemia is acute myeloid leukemia (AML), and the acute myeloid leukemia is primary acute myeloid leukemia. In an exemplary embodiment, the leukemia is acute myeloid leukemia (AML), and the acute myeloid leukemia is secondary acute myeloid leukemia. In an exemplary embodiment, the leukemia is erythroleukemia. In an exemplary embodiment, the leukemia is eosinophilic leukemia. In an exemplary embodiment, the leukemia is acute myeloid leukemia (AML), and the acute myeloid leukemia does not include acute promyelocytic leukemia. In an exemplary embodiment, the leukemia is acute myeloid leukemia (AML), and the acute myeloid leukemia is blastic plasmacytoid dendritic cell neoplasm. In an exemplary embodiment, the leukemia is B-cell acute lymphocytic leukemia (B-ALL). In an exemplary embodiment, the leukemia is T-cell acute lymphocytic leukemia (T-ALL).

### Dosage Regimen

In some embodiments, the bispecific anti-CD123 x anti-CD3 antibody (e.g., XENP14045) is administered according to a dosage regimen described herein. Dosage regimens are adjusted to provide the optimum desired response (e.g., a therapeutic response). The efficient dosages and the dosage regimens for the bispecific anti-CD123 xCD3 antibodies used in the present invention depend on the disease or condition to be treated and may be determined by the persons skilled in the art.

In an exemplary embodiment, the bispecific anti-CD123 x anti-CD3 antibody (e.g., XENP14045) is administered intravenously by infusion once every 6-8 days in an amount of from about 1 ng/kg to about 800 ng/kg.

In an exemplary embodiment, the bispecific anti-CD123 x anti-CD3 antibody (e.g., XENP14045) is administered intravenously by infusion monthly in an amount of from about 30 ng/kg to about 750 ng/kg, e.g., about 75 ng/kg to about 750 ng/kg, about 75 ng/kg to about 700 ng/kg, about 75 ng/kg to about 650 ng/kg, about 75 ng/kg to about 600 ng/kg, about 75 ng/kg to about 550 ng/kg, about 75 ng/kg to about 500 ng/kg, about 75 ng/kg to about 450 ng/kg, about 75 ng/kg to about 400 ng/kg, about 75 ng/kg to about 350 ng/kg, about 75 ng/kg to about 300 ng/kg, about 75 ng/kg to about 250 ng/kg, about 75 ng/kg to about 200 ng/kg, about 75 ng/kg to about 150 ng/kg, or about 75 ng/kg to about 100 ng/kg.

In an exemplary embodiment, the bispecific anti-CD123 x anti-CD3 antibody (e.g., XENP14045) is administered intravenously by infusion every other week in an amount of from about 30 ng/kg to about 750 ng/kg, e.g., about 75 ng/kg to about 750 ng/kg, about 75 ng/kg to about 700 ng/kg, about 75 ng/kg to about 650 ng/kg, about 75 ng/kg to about 600 ng/kg, about 75 ng/kg to about 550 ng/kg, about 75 ng/kg to about 500 ng/kg, about 75 ng/kg to about 450 ng/kg, about 75 ng/kg to about 400 ng/kg, about 75 ng/kg to about 350 ng/kg, about 75 ng/kg to about 300 ng/kg, about 75 ng/kg to about 250 ng/kg, about 75 ng/kg to about 200 ng/kg, or about 75 ng/kg to about 150 ng/kg, or about 75 ng/kg to about 100 ng/kg.

In an exemplary embodiment, the bispecific anti-CD123 x anti-CD3 antibody (e.g., XENP14045) is administered by infusion for a period of between about one hour and about three hours. In an exemplary embodiment, the bispecific anti-CD123 x anti-CD3 antibody (e.g., XENP14045) is administered by infusion for a period of about two hours. In an exemplary embodiment, the bispecific anti-CD123 x anti-CD3 antibody (e.g., XENP14045) is administered by infusion for a period of two hours.

In an exemplary embodiment, the bispecific anti-CD123 x anti-CD3 antibody (e.g., XENP14045) is administered once every 6-8 days for between about 1 and about 9 weeks. In an exemplary embodiment, the bispecific anti-CD123 x anti-CD3 antibody (e.g., XENP14045) is administered once every 6-8 days for between about 2 and about 7 weeks. In an exemplary embodiment, the bispecific anti-CD123 x anti-CD3 antibody (e.g., XENP14045) is administered once every 6-8 days for between about 3 and about 9 weeks. In an exemplary embodiment, the bispecific anti-CD123 x anti-CD3 antibody (e.g., XENP14045) is administered once every 6-8 days for between about 1 and about 8 weeks. In an exemplary embodiment, the bispecific anti-CD123 x anti-CD3 antibody (e.g., XENP14045) is administered once every 6-8 days for between about 3 and about 5 weeks. In an exemplary embodiment, the bispecific anti-CD123 x anti-CD3 antibody (e.g., XENP14045) is administered once every 6-8 days for about 4 weeks. In an exemplary embodiment, the bispecific anti-CD123 x anti-CD3 antibody (e.g., XENP14045) is administered once every 6-8 days for 4 weeks. In an exemplary embodiment, the bispecific anti-CD123 x anti-CD3 antibody (e.g., XENP14045) is administered once every 6-8 days for between about 7 and about 9 weeks. In an exemplary embodiment, the bispecific anti-CD123 x anti-CD3 antibody (e.g., XENP14045) is administered once every 6-8 days for about 8 weeks. In an exemplary embodiment, the bispecific anti-CD123 x anti-CD3 antibody (e.g., XENP14045) is administered once every 6-8 days for 8 weeks.

The dosage may be determined or adjusted by measuring the amount of bispecific anti-CD123 x anti-CD3 antibody (e.g., XENP14045) of the present invention in the blood upon administration using techniques known in the art, for instance taking out a biological sample and using anti-idiotypic antibodies which target the antigen binding region of the bispecific anti-CD123 x anti-CD3 antibody (e.g., XENP14045).

In an exemplary embodiment, the amount is between about 3 ng/kg and about 750 ng/kg.

In an exemplary embodiment, the amount is between about 30 ng/kg and about 750 ng/kg. In an exemplary embodiment, the amount is between about 75 ng/kg and about 750 ng/kg.

In an exemplary embodiment, the amount is between about 1 ng/kg and about 5 ng/kg. In an exemplary embodiment, the amount is between about 2 ng/kg and about 4 ng/kg. In an exemplary embodiment, the amount is about 3 ng/kg. In an exemplary embodiment, the amount is 3 ng/kg.

In an exemplary embodiment, the amount is between about 1 ng/kg and about 20 ng/kg. In an exemplary embodiment, the amount is between about 5 ng/kg and about 15 ng/kg. In an exemplary embodiment, the amount is between about 7 ng/kg and about 13 ng/kg. In an exemplary embodiment, the amount is between about 9 ng/kg and about 11 ng/kg. In an exemplary embodiment, the amount is about 10 ng/kg. In an exemplary embodiment, the amount is 10 ng/kg.

In an exemplary embodiment, the amount is between about 10 ng/kg and about 50 ng/kg. In an exemplary embodiment, the amount is between about 20 ng/kg and about 40 ng/kg. In an exemplary embodiment, the amount is between about 25 ng/kg and about 35 ng/kg. In an exemplary embodiment, the amount is about 30 ng/kg. In an exemplary embodiment, the amount is 30 ng/kg.

In an exemplary embodiment, the amount is between about 25 ng/kg and about 150 ng/kg. In an exemplary embodiment, the amount is between about 50 ng/kg and about 125 ng/kg. In an exemplary embodiment, the amount is between about 50 ng/kg and about 100 ng/kg. In an exemplary embodiment, the amount is between about 55 ng/kg and about 95 ng/kg. In an exemplary embodiment, the amount is between about 60 ng/kg and about 90 ng/kg. In an exemplary embodiment, the amount is between about 65 ng/kg and about 85 ng/kg. In an exemplary embodiment, the amount is between about 70 ng/kg and about 80 ng/kg. In an exemplary embodiment, the amount is about 75 ng/kg. In an exemplary embodiment, the amount is 75 ng/kg.

In an exemplary embodiment, the amount is between about 50 ng/kg and about 250 ng/kg. In an exemplary embodiment, the amount is between about 75 ng/kg and about 225 ng/kg. In an exemplary embodiment, the amount is between about 100 ng/kg and about 200 ng/kg. In an exemplary embodiment, the amount is between about 125 ng/kg and about 175 ng/kg. In an exemplary embodiment, the amount is about 150 ng/kg. In an exemplary embodiment, the amount is 150 ng/kg.

In an exemplary embodiment, the amount is between about 100 ng/kg and about 500 ng/kg. In an exemplary embodiment, the amount is between about 200 ng/kg and about 400 ng/kg. In an exemplary embodiment, the amount is between about 200 ng/kg and about 400 ng/kg. In an exemplary embodiment, the amount is between about 225 ng/kg and about 375 ng/kg. In an exemplary embodiment, the amount is between about 250 ng/kg and about 350 ng/kg. In an exemplary embodiment, the amount is between about 275 ng/kg and about 325 ng/kg. In an exemplary embodiment, the amount is about 300 ng/kg. In an exemplary embodiment, the amount is 300 ng/kg.

In an exemplary embodiment, the amount is between about 350 ng/kg and about 650 ng/kg. In an exemplary embodiment, the amount is between about 400 ng/kg and about 600 ng/kg. In an exemplary embodiment, the amount is between about 450 ng/kg and about 550 ng/kg. In an exemplary embodiment, the amount is between about 475 ng/kg and about 525 ng/kg. In an exemplary embodiment, the amount is about 500 ng/kg. In an exemplary embodiment, the amount is 500 ng/kg.

In an exemplary embodiment, the amount is between about 600 ng/kg and about 900 ng/kg. In an exemplary embodiment, the amount is between about 650 ng/kg and about 850 ng/kg. In an exemplary embodiment, the amount is between about 700 ng/kg and about 800 ng/kg. In an exemplary embodiment, the amount is between about 725 ng/kg and about 775 ng/kg. In an exemplary embodiment, the amount is about 750 ng/kg. In an exemplary embodiment, the amount is 750 ng/kg.

In some embodiments, the bispecific anti-CD123 x anti-CD3 antibody (e.g., XENP14045) is administered intravenously. In some embodiments, the bispecific anti-CD123 x anti-CD3 antibody (e.g., XENP14045) is administered weekly until disease progression, unacceptable toxicity, or individual choice.

In some embodiments, the bispecific anti-CD123 x anti-CD3 antibody (e.g., XENP14045) is a front line therapy, second line therapy, third line therapy, fourth line therapy, fifth line therapy, or sixth line therapy.

In some embodiments, the bispecific anti-CD123 x anti-CD3 antibody (e.g., XENP14045) treats a refractory leukemia. In some embodiments, the bispecific anti-CD123 x anti-CD3 antibody (e.g., XENP14045) is a maintenance therapy.

A medical professional having ordinary skill in the art may readily determine and prescribe the effective amount of the antibody composition required. For example, a physician could start doses of the medicament employed in the antibody composition at levels lower than that required in order to achieve the desired therapeutic effect and gradually increase the dosage until the desired effect is achieved.

### Treatment Modalities

In the methods of the invention, treatment is used to provide a positive therapeutic response with respect to a leukemia. By “positive therapeutic response” is intended an improvement in the leukemia, and/or an improvement in the symptoms associated with the leukemia. For example, a positive therapeutic response would refer to one or more of the following improvements in the leukemia: (1) a reduction in the number of CD123⁺ leukemia-associated cells, including CD123⁺peripheral blood basophils and/or marrow basophils; (2) an increase in CD123⁺leukemia-associated cell death; (3) inhibition of CD123⁺leukemia-associated cell survival; (5) inhibition (i.e., slowing to some extent, preferably halting) of CD123⁺cell proliferation; (6) an increased patient survival rate; and (7) some relief from one or more symptoms associated with the leukemia.

Positive therapeutic responses in any given leukemia can be determined by standardized response criteria specific to that leukemia.

In addition to these positive therapeutic responses, the subject undergoing treatment may experience the beneficial effect of an improvement in the symptoms associated with the leukemia. In an exemplary embodiment, a treatment of leukemia is selected from the group consisting of feeling less tired, feeling less weak, feeling less dizzy or lightheaded, reduction in shortness of breath, reduction in fever, quicker response to infections, reduction in ease of bruising, reduction in bleeding episodes, weight gain, reduction in night sweats, gain of appetite, reduction in abdominal swelling, reduction in lymph node swelling, reduction in bone or joint pain, and reduction in thymus swelling.

An improvement in the leukemia may be characterized as a complete response. By “complete response” is intended an absence of clinically detectable disease with normalization of any previously abnormal radiographic studies, bone marrow, and cerebrospinal fluid (CSF) or abnormal monoclonal protein in the case of myeloma.

Such a response may persist for at least 4 to 8 weeks, or sometimes 6 to 8 weeks, following treatment according to the methods of the invention. Alternatively, an improvement in the leukemia may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody are outweighed by the therapeutically beneficial effects.

A “therapeutically effective amount” for therapy may also be measured by its ability to stabilize the progression of the leukemia. The ability of an antibody to inhibit leukemia may be evaluated in an animal model system predictive of efficacy in a human.

Alternatively, this property of an antibody composition may be evaluated by examining the ability of the antibody to inhibit cell growth or to induce apoptosis by in vitro assays known to the skilled practitioner. A therapeutically effective amount of a bispecific anti-CD123 x anti-CD3 antibody (e.g., XENP14045) reduce the number of CD123⁺ leukemia-associated cells, or improve other aspects related to the leukemia (such as those described herein), and/or otherwise ameliorate symptoms in a human subject (such as those also described herein). One of ordinary skill in the art would be able to determine such amounts based on such factors as the subject's size, the severity of the subject's symptoms, and the particular antibody composition or route of administration selected.

### Combination Therapy

In certain instances, a bispecific anti-CD123 x anti-CD3 antibody (e.g., XENP14045) described herein can be used in combination with another therapeutic agent. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The bispecific anti-CD123 x anti-CD3 antibody (e.g., XENP14045) described herein and the at least one additional therapeutic agent can be administered simultaneously, in the same or in separate compositions, or sequentially. For sequential administration, the bispecific anti-CD123 x anti-CD3 antibody (e.g., XENP14045) described herein can be administered first, and the additional agent can be administered second, or the order of administration can be reversed.

The bispecific anti-CD123 x anti-CD3 antibody (e.g., XENP14045) and/or other therapeutic agents, procedures or modalities can be administered during periods of active disorder, or during a period of remission or less active disease. The bispecific anti-CD123 x anti-CD3 antibody (e.g., XENP14045) can be administered before the other treatment, concurrently with the treatment, post-treatment, or during remission of the disorder.

When administered in combination, the bispecific anti-CD123 x anti-CD3 antibody (e.g., XENP14045) and the additional agent (e.g., second or third agent), or all, can be administered in an amount or dose that is higher, lower or the same than the amount or dosage of each agent used individually, e.g., as a monotherapy. In some embodiments, the administered amount or dosage of the bispecific anti-CD123 x anti-CD3 antibody (e.g., XENP14045), the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the bispecific anti-CD123 x anti-CD3 antibody (e.g., XENP14045), the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a bispecific anti-CD123 x anti-CD3 antibody (e.g., XENP14045) described herein may be used in a treatment regimen in combination with chemotherapy, radiation, immunosuppressive agents, such as cyclosporin, azathioprine, methotrexate, mycophenolate, and FK506, antibodies, or other immunoablative agents such as CAMPATH, other antibody therapies, cytoxan, fludarabine, cyclosporin, FK506, rapamycin, mycophenolic acid, steroids, FR90165, cytokines, and irradiation. peptide vaccine, such as that described in Izumoto et al. 2008 J Neurosurg 108:963-971.

In certain instances, compounds of the present invention are combined with other therapeutic agents, such as other anti-cancer agents, anti-allergic agents, anti-nausea agents (or anti-emetics), pain relievers, cytoprotective agents, and combinations thereof.

In one embodiment, a bispecific anti-CD123 x anti-CD3 antibody (e.g., XENP14045) described herein can be used in combination with a chemotherapeutic agent. Exemplary chemotherapeutic agents include an anthracycline (e.g., idarubicin, daunorubicin, doxorubicin (e.g., liposomal doxorubicin)), a anthracenedione derivative (e.g., mitoxantrone), a vinca alkaloid (e.g., vinblastine, vincristine, vindesine, vinorelbine), an alkylating agent (e.g., cyclophosphamide, dacarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, cytarabine, pyrimidine analogs, purine analogs and adenosine deaminase inhibitors (e.g., fludarabine)), an mTOR inhibitor, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex), capecitabine (Xeloda) N4-pentoxycarbonyl-5-deoxy-5-fluorocytidine, carboplatin (Paraplatin ®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar) L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, a bispecific anti-CD123 x anti-CD3 antibody (e.g., XENP14045) described herein is administered to a subject in combination with one or more of the following agents: an anti-INF antibody, a steroid, or an antirnslarnine (e. Benadryl).

In some embodiments, a bispecific anti-CD123 x anti-CD3 antibody (e.g., XENP14045) described herein is administered to a subject who has ALL, in combination with one or more of the following agents: Methotrexate (e.g., Abitrexate, Methotrexate LPF, Mexate, Mexate-AQ, Folex, Folex PFS), Nelarabine (e.g., Arranon), Doxorubicin Hydrochloride, Daunorubicin Hydrochloride (e.g., Cerubidine, Rubidomycin) (in combination with cytarabine and anthracycline-daunorubicin or idararubicin), Clofarabine (e.g., Clofarex or Clolar), Cyclophosphamide (e.g., Cytoxan, Neosar, Clafen), Cytarabine (e.g., Cytosar-U, Tarabine PFS), Dasatinib (e.g., Sprycel), other Brc tyrosine kinase inhibitor, Erwinaze (e.g., Asparaginase Erwinia Chrysanthemi), Imatinib Mesylate (e.g., Gleevec), Ponatinib Hydrochloride (e.g., Iclusig), https://www.cancer.gov/about-cancer/treatment/drugs/vincristine-sulfate-liposomeMercaptopurine (e.g., Purinethol, Purixan), Pegaspargase (e.g., Oncaspar), Ponatinib Hydrochloride, Prednisone, Vincristine Sulfate, Vincristine Sulfate Liposome (e.g., Marqibo), Vincasar PFS, Hyper-CVAD.

In some embodiments, a bispecific anti-CD123 x anti-CD3 antibody (e.g., XENP14045) described herein is administered to a subject who has AML, in combination with one or more of the following agents: Daunorubicin Hyrdochloride (e.g., Cerubidine or Rubidomycin) (optionally in combination with cytarabine and anthracycline-daunorubicin or idararubicin), Idarubicin Hydrochloride (e.g., Idamycin), BCL2 inhibitor (e.g., Venclextra), Cyclophosphamide (e.g., Cytoxan, Clafen, Neosar), Cytarabine (e.g., Cytosar-U, Tarabine PFS), Doxorubicin Hydrochloride, Decitabine (hypomethylating agent), Fludarabine (fludara), Flt3 inhibitors (e.g., sunitinib, sorafenib, midostaurin, lestaurtinib, quizartinib, crenolanib, PLX3397), GCSF (Granulocyte-colony stimulating factor), IDH inhibitors (e.g., IDH1 inhibbitors, e.g., AG120 or IDH305); IDH2 inhibitors, e.g., AG221; pan IGH1/IGH2 inhibitors, e.g., AG881), Mitoxantrone Hydrochloride, Thioguanine (e.g., Tabloid), azacitidine (e.g., Vidaza, hypomethylating agent), Vincristine Sulfate (e.g., Vincasar PFS).

In some embodiments, a bispecific anti-CD123 x anti-CD3 antibody (e.g., XENP14045) described herein is administered to a subject who has CML, in combination with one or more of the following agents: Bosutinib (e.g., Bosulif), Busulfan (e.g., Busulfex), Cyclophosphamide (e.g., Clafen, Cytoxan, Neosar), Cytarabine (e.g., Cytosar-U, Tarabine PFS), Dasatinib (e.g., Sprycel), Imatinib Mesylate (e.g., Gleevec), Hydroxyurea (e.g., Hydrea), Ponatinib Hydrochloride (e.g., Iclusig), Mechlorethamine Hydrochloride (e.g., Mustargen), Busulfan (e.g., Myleran), Nilotinib, Omacetaxine Mepesuccinate (e.g., Synribo).

In one embodiment, the subject can be administered an agent which reduces or ameliorates a side effect associated with the administration of a bispecific anti-CD123 x anti-CD3 antibody (e.g., XENP14045). Side effects associated with the administration of a bispecific anti-CD123 x anti-CD3 antibody (e.g., XENP14045) include, but are not limited to, cytokine release syndrome (“CRS”) and hemophagocytic lymphohistiocytosis (HLH), also termed Macrophage Activation Syndrome (MAS). Symptoms of CRS may include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symsptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardac output (early) and potentially diminished cardiac output (late). CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures.

Accordingly, the methods described herein can comprise administering a bispecific anti-CD123 x anti-CD3 antibody (e.g., XENP14045) described herein to a subject and further administering one or more agents to manage elevated levels of a soluble factor resulting from treatment with a bispecific anti-CD123 x anti-CD3 antibody (e.g., XENP14045). In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and inhibitor of IL-1R, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitor of TNFα include, but are not limited to, xanthine derivatives (e.g. pentoxifylline) and bupropion. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab. An example of an IL-1R based inhibitor is anakinra.

In some embodiment, the subject is administered a corticosteroid, such as, e.g., methylprednisolone, hydrocortisone, among others. In some embodiments, the subject is administered a corticosterioid, e.g., methylprednisolone, hydrocortisone, in combination with Benadryl and Tylenol prior to the administration of a anti-CD123 x anti-CD3 antibody (e.g., XENP14045) to mitigate the CRS risk.

In some embodiments, the subject is administered a vasopressor, such as, e.g., norepinephrine, dopamine, phenylephrine, epinephrine, vasopressin, or a combination thereof.

In an embodiment, the subject can be administered an antipyretic agent. In an embodiment, the subject can be administered an analgesic agent.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, and 2014/0288275, as well as PCT Publication WO2014/145806, as well as U.S. Applications 62/085,027, Ser. Nos. 14/952,714, and 15/141,350, all of which are expressly incorporated by reference in their entirety and particularly for the techniques outlined therein.

### Example 1

### XENP14045 Treatment Plan

This is a multicenter, open-label, multi-dose, single-arm, Phase 1, dose-escalation study of XENP14045. The dose of XENP14045 will be administered IV over a 2-hr infusion period. Modifications of the dose infusion period may occur based on any observed infusion toxicity.

This study will be conducted in 2 sequential parts, Parts A and B.

Part A: Patients will be enrolled in up to 8 consecutive dose cohorts (0.003, 0.01, 0.03, 0.075, 0.15, 0.3, 0.5, and 0.75 μg/kg) with initial accelerated titration for the first 3 cohorts. The first 3 cohorts will consist of 1 patient each until there is evidence of a Grade 2 toxicity, and the remaining cohorts will enroll at least 3 patients each in a classic 3+3 dose escalation scheme. Patients will be admitted for 3 days for the first and fourth doses (and 2 days for the second dose, if admission is necessary to collect cytokine/inflammatory factors for the 8 hr postinfusion timepoint) for observation, PK, PD, and laboratory assessment. Within each ascending dose cohort (Cohorts 1A-8A), patients will be given XENP14045 IV over 2 hr, once every 7 days, for a total of 4 doses in each 28-day cycle. The initial treatment period will include 2 cycles. After the MTD and/or RD dose is reached, the cohort may be expanded by up to an additional 12 patients to obtain additional safety data.

Part B: An attempt will be made to escalate to higher doses for the second and subsequent drug infusions. Patients will be admitted for 3 days for the first and fourth dose as in Part A, but also for the escalated second dose (Day 8) for observation, PK, PD, and cytokine assessment.

The dose to be administered to the patient for all cohorts will be calculated based on baseline (Day-1) weight measurement in kg. Following the first dose, subsequent doses will only be modified if the patient's weight changes by more than 10% from the Day-1 weight at which point it will be recalculated using the current weight. For patients whose weight exceeds 100 kg, the dose of XENP14045 will be calculated based on a weight of 100 kg and will NOT be calculated based upon the patient's actual body weight.

A dose escalation schema will be employed in single dose level cohorts for Part A and sequentially increasing second and subsequent infusion dosing cohorts for Part B. Dose escalation will continue in both Parts A and B until the MTD and/or RD for further study has been identified or until a dose of 0.75 μg/kg has been reached, whichever comes first.

Patients will receive two 28-day cycles (8 weekly doses) of therapy. In the absence of unacceptable study drug-related toxicity, patients may receive additional cycles of therapy if there is clinical benefit (as assessed by the investigator). Doses will be administered on Days 1, 8, 15, and 22 of each cycle. Dosing may be delayed in the presence of drug-related toxicities. DLT determination and safety evaluation will occur after all relevant data is available through Day 22 of Cycle 1. If the MTD and/or RD are not reached, dose escalation to the next dose cohort will occur. Patients will be followed for at least 4 weeks after treatment is discontinued. Information regarding disease status will be collected by the investigational sites up to a final dose of XENP14045, and followed by either clinic visit or telephone contact for an additional 6 months, or until the occurrence of death, stem cell transplantation, or disease progression requiring therapy (whichever comes first).

### Dose Escalation Scheme Part A

In Part A, dose level increases will initially proceed according to an accelerated titration design (see Table 1). This design allows for more efficient dose escalation while maintaining safety standards by implementing conservative triggers for cohort expansion during the accelerated escalation phase, and may limit the number of patients exposed to potentially sub-therapeutic doses of XENP14045.

TABLE 1
Study Cohorts - Part A

| | Cohort | Planned Dose | | Patients | |
|---|---|---|---|---|---|
| Part A | 1A | 3 ng/kg | (0.003 μg/kg) | 1 | (+2 + 3) |
| | 2A | 10 ng/kg | (0.01 μg/kg) | 1 | (+2 + 3) |
| | 3A | 30 ng/kg | (0.03 μg/kg) | 1 | (+2 + 3) |
| | 4A | 75 ng/kg | (0.075 μg/kg) | 3 | (+3) |
| | 5A | 150 ng/kg | (0.150 ug/kg) | 3 | (+3) |
| | 6A | 300 ng/kg | (0.3 μg/kg) | 3 | (+3) |
| | 7A | 500 ng/kg | (0.5 μg/kg) | 3 | (+3) |
| | 8A | 750 ng/kg | (0.75 μg/kg) | 3 | (+3) |

| | | |
|---|---|---|
| Expansion-A | At MTD or recommended | Up to 12 |
| | first infusion dose | |

During the initial accelerated dose escalation phase (Cohorts 1A, 2A, and 3A), dose escalation may occur after treatment of 1 patient per cohort provided that there is no≧Grade 2 toxicity during Cycle 1 and the patient has met minimum safety assessment requirements (see Table 2). When a patient experiences a≧Grade 2 toxicity during the dose escalation safety assessment period, the accelerated escalation phase will end, the standard dose escalation phase will begin, and the cohort in which the event(s) occurred will be expanded to a total of at least 3 patients (2 additional patients will be enrolled).

TABLE 2
Dose Escalation Scheme
Accelerated Dose Escalation Phase

| | Number of Patients | |
|---|---|---|
| | Enrolled and Assessable | |
| Number of Patients | for Safety Following | |
| with at Least One | Four Doses of | |
| Event ≧ Grade 2 | XENP14045 | Escalation Decision |
| 0 | 1 | Escalate to the next |
| | | higher dose level |
| 1 | 1 | Enroll 2 additional |
| | | patients on the same |
| | | dose level and revert |
| | | to Standard Dose |
| | | Escalation (3 + 3) |
| | | design below. |

Standard Dose Escalation Phase

| | Number of Patients | |
|---|---|---|
| | Enrolled and Assessable | |
| Number of Patients | for Safety Following | |
| with at Least One | Four Doses of | |
| DLT | XENP14045 | Escalation Decision |
| 0 | 3 | Escalate to the next |
| | | higher dose level |
| 1 | 3 | Enroll 3 additional |
| | | patients on the |
| | | same dose level |
| 1 | 6 | Escalate to the next |
| | | higher dose level |
| 2 | 3 or 6 | No dose escalation may |
| | | occur; MTD has been |
| | | surpassed. The next |
| | | lower dose level should |
| | | be expanded. |

DLT = dose-limiting toxicity;
MTD = maximum tolerated dose

From this cohort forward (or beginning with Cohort 4A [0.075 μg/kg], whichever comes first) the standard 3+3 dose escalation rules will apply:

If zero of 3 patients have a DLT, then dose escalation to the next level will occur.

If 1 of 3 patients has a DLT, then the cohort will be further expanded to a total of 6 patients or until a second patient in the cohort experiences a DLT. If there are no additional patients with a DLT, then dose escalation to the next higher dose level will occur.

The MTD is defined as the highest dose level at which no more than 1 patient experiences DLT out of 6 patients assessable for toxicity at that dose level. Any cohort with 2 or more patients experiencing a DLT will have exceeded the MTD and there will be no further dose escalation. The dose level below the cohort at which 2 or more patients with DLT occurred will be expanded to at least 6 to delineate the MTD.

Before a dose-escalation decision can be reached, at least 1 patient (in the accelerated dose escalation phase of the study) or 3 patients (in the standard escalation phase of the study) must meet all requirements for dose escalation safety assessment.

For the purpose of determining the incidence of DLT and defining the MTD and/or recommended dosing of XENP14045 for future study, only patients who experience DLT and those with sufficient safety data/follow-up will be evaluated. Patients who complete 4 doses of XENP14045 and undergo the planned safety evaluations through Day 22 will be considered to have sufficient safety data/follow-up. Patients who withdraw from study before completing Day 22 of treatment for reasons unrelated to study drug toxicity will be considered to have inadequate data to support dose escalation. In such cases, replacement patients will be enrolled to receive the same dose of XENP14045 as the patients who withdraw prematurely.

Once the MTD (or RD for further study) is identified, the MTD/RD dose level may be further expanded up to an additional 12 patients (up to a total MTD/RD cohort of 18 patients) to further assess safety and PK.

The dose escalation scheme may be modified (e.g., smaller increases or decreases in dose level may be permitted, additional patients in a cohort may be enrolled, infusion duration and scheduling may be modified) based on the type and severity of toxicities observed in this trial, upon agreement of the DERC. Enrolling additional patients beyond 66 requires a protocol amendment.

### Dose Escalation Scheme-Part B

In Part B, the Day 1 dose will be fixed at the level determined in Part A. The second dose will be escalated and maintained for subsequent doses. Dosing cohorts will be defined relative to the MTD/RD determined in Part A.

TABLE 3
Study Cohorts- Part B

| | Cohort | Day 1 | Day 8 | Day 15 | Day 22 | Patients |
|---|---|---|---|---|---|---|
| Part B | −1B | X | X | X + 1 | X + 1 | 3 (+3) |
| | 1B | X | X + 1 | X + 1 | X + 1 | 3 (+3) |
| | 2B | X | X + 2 | X + 2 | X + 2 | 3 (+3) |
| | 3B | X | X + 3 | X + 3 | X + 3 | 3 (+3) |
| | 4B | X | X + 4 | X + 4 | X + 4 | 3 (+3) |
| | 5B | X | X + 5 | X + 5 | X + 5 | 3 (+3) |
| | 6B | X | X + 6 | X + 6 | X + 6 | 3 (+3) |
| | 7B | X | X + 7 | X + 7 | X + 7 | 3 (+3) |

| | | |
|---|---|---|
| Expansion-B | At MTD or RD cohort | Up to 12 |

MTD = maximum tolerated dose;
RD = recommended dose;
X = Part A MTD/RD

Dose escalation will proceed as described for the standard 3+3 scheme noted in Part A and with the same dosing levels (0.003, 0.01, 0.03, 0.075, 0.15, 0.3, 0.5, and 0.75 μg/kg) however the Day 1 infusion dose will always be the MTD/RD determined in Part A (denoted as “X” in Table 3). Dose escalation on each Part B cohort will be based on this starting point so for example if the MTD/RD from Part A is 0.03 μg/kg, the first infusion in Cohort 1B will be 0.03 μg/kg and the second and subsequent infusions will be at 0.075 μg/kg (i.e. X+1).

A minimum of 3 patients will be enrolled in each cohort. As in Part A, no two patients will start treatment with XENP14045 on the same day. If all 3 patients tolerate a cohort without experiencing DLT (and the DERC agrees), enrollment will begin on the next higher cohort. If at any time through Day 22 a DLT occurs, 3 additional patients will be added to the cohort. If there is an additional DLT among the 6 patients on the cohort, the previous dosing cohort will be expanded to 6 to establish a MTD and/or RD. If this occurs on cohort 1B, the next 3 patients will be enrolled on cohort -1B. If there are no further DLTs among the 3 additional patients, another 3 patients will be added to the cohort. If there is an additional DLT, then the MTD/RD and schedule established in Part A will be recommended for further study.

### Example 2

### In Vitro Antitumor Efficacy

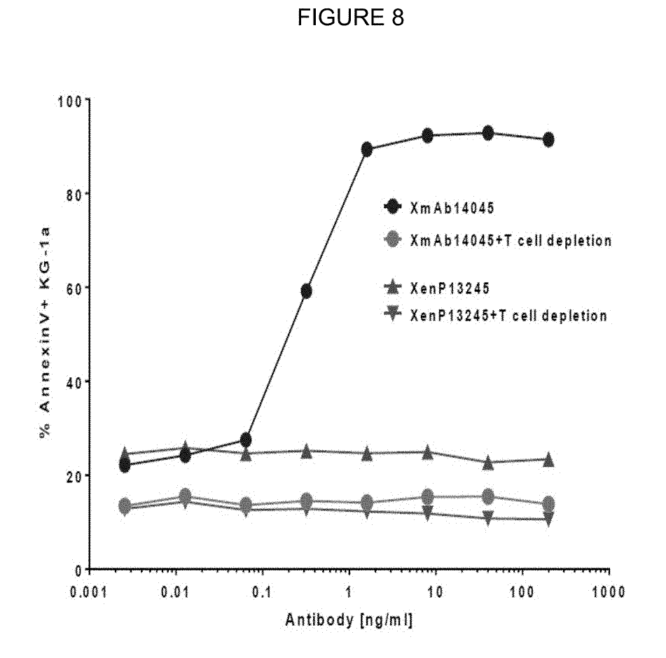

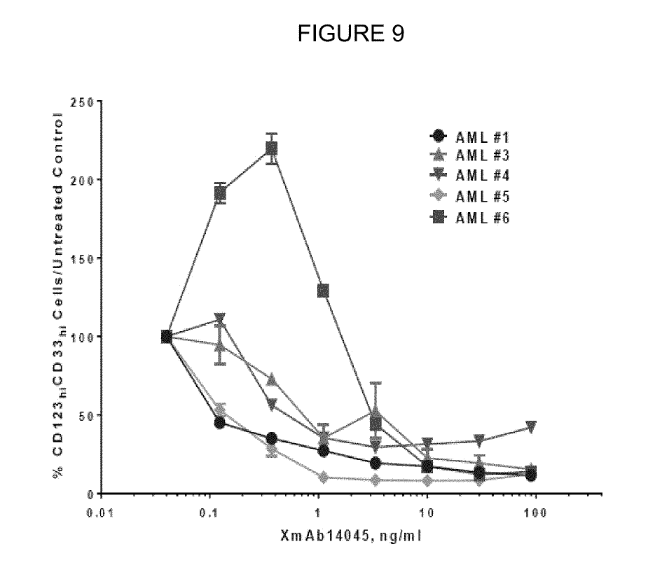

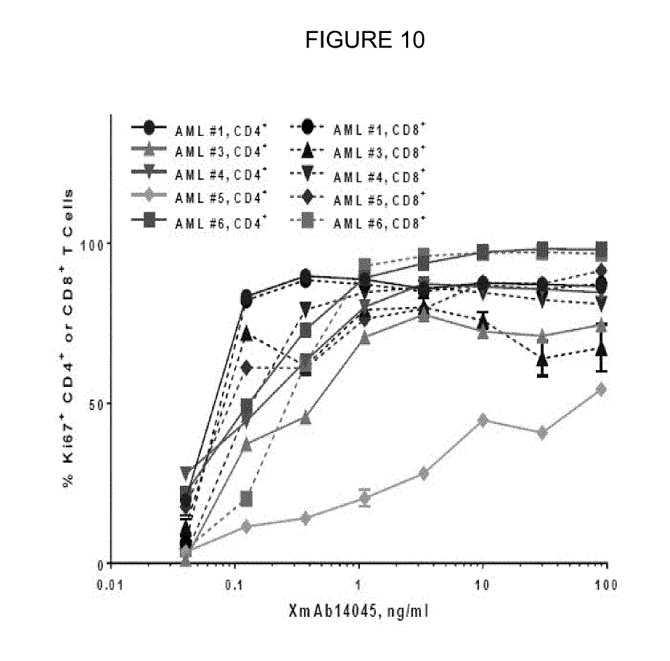

T cell-dependent cytotoxicity of XmAb14045 against CD123-positive (KG1a and Kasumi-3) and CD123-negative (Ramos) cell lines was examined using purified PBMC or T cell-depleted PBMC as effector cells. In addition, T cell activation was assessed by quantifying CD69 induction (a marker of lymphocyte activation) on both CD4+and CD8+ T cells. XENP13245, an anti-RSV x anti-CD3 bsAb, was used as a control. XmAb14045, but not XENP13245, showed robust and potent killing of the CD123⁺ KG-1a (EC₅₀ of 0.28 ng/mL; see FIG. 8) and Kasumi-3 (EC₅₀ of 0.01 ng/mL) cell lines when supplied with human PBMC as an effector population along with robust CD69 induction in both CD4⁺ and CD8⁺ T cells. However, when T cells were depleted from PBMC (FIG. 8), XmAb14045 failed to induce killing or induce CD69 expression on T cells. XmAb14045 did not induce cytotoxicity of the CD123⁻ Ramos B cell line or induce T cell activation as measured by CD69 expression.

A series of studies was performed to evaluate the functionality of T-cells derived from AML patient-derived PBMC. In particular, the ability of XmAb14045 to mediate RTCC towards various target populations found within, or added to, the AML samples was investigated. The target populations included: 1) a CD123ₕᵢCD33ₕᵢ population that arises in both AML PBMC and healthy PBMC upon incubation in culture for several days; 2) putative AML blast cells identified in the samples by flow cytometry; and 3) added KG1a AML cells. CD123-dependent T cell activation was measured by CD25 and Ki-67 upregulation on T cells. CD123-dependent target cell killing was monitored using annexin-V staining and by monitoring the reduction of counted blast cells.

Multiple AML patient PBMC and normal PBMC samples were tested for XmAb14045-induced target cell killing and T cell activation. Both AML and normal PBMC contained CD123_{high} and CD33_{high} (CD123ₕᵢCD33ₕᵢ) cells; therefore, this population likely does not represent leukemic blast cells, but does serve as a useful surrogate target population. After 6 days incubation of PBMCs with XmAb14045, dose-dependent partial depletion of CD123ₕᵢCD33ₕᵢ cells was induced in AML patient-derived PBMC, accompanied by CD4⁺ and CD8⁺ T cell activation and proliferation.

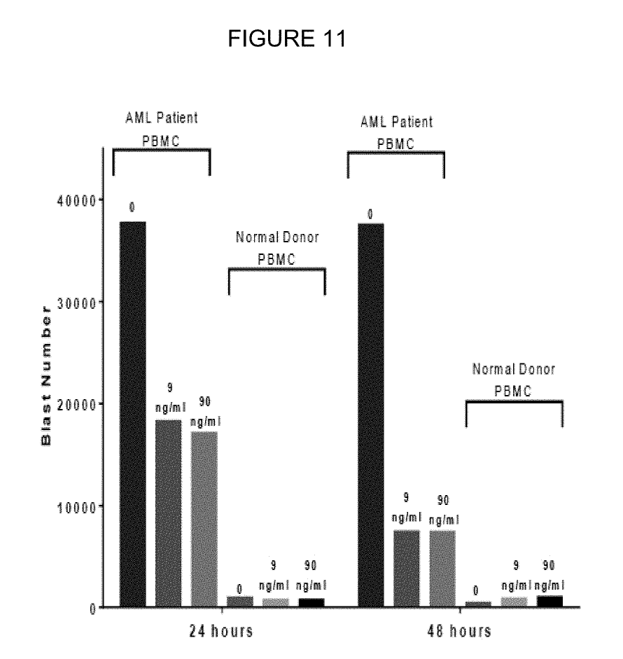

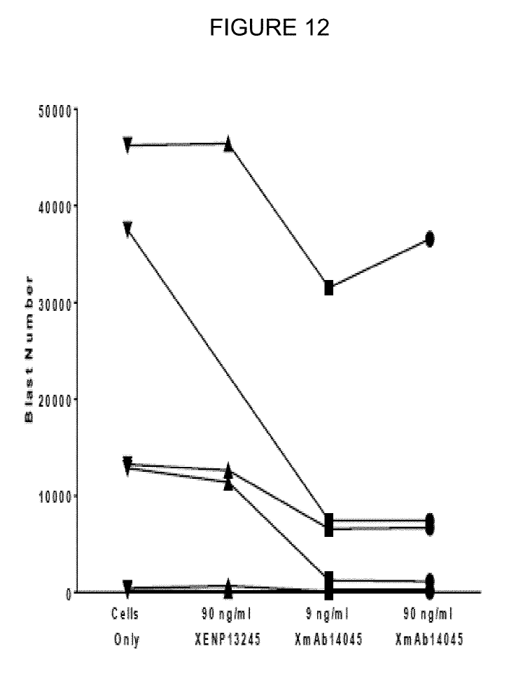

In a second set of studies, a modified staining process was used to detect leukemic blast cells in PBMC from a patient with AML. AML PBMCs or PBMCs from a normal control donor were incubated for 24 or 48 hours with XmAb14045 at concentrations of 9 or 90 ng/mL and the putative blast cell number was obtained by flow cytometry. XmAb14045 reduced blast number by approximately 80% at 48 hours (FIG. 11). As expected, no blasts were seen in the normal donor PBMCs. This result was extended by assessing a total of 6 AML patients. XmAb14045 at concentrations of 9 or 90 ng/mL or XENP13245 (anti-RSV x anti-CD3) as a negative control. XmAb14045 depleted this putative blast cell population in AML PBMC at 48 hours by approximately 20% to 90%, with no apparent dependence on the number of target cells or T cells in the samples (see FIG. 12). The depletion was again associated with activation and proliferation of T cells.

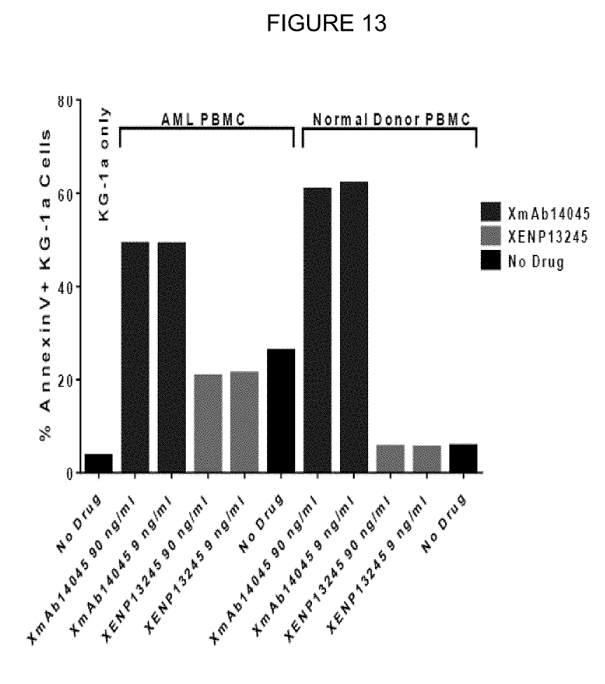

In a third set of studies, killing of an AML tumor cell line by AML patient T cells was assessed. PBMC from one AML donor was mixed with the CD123-expressing cell line KG-1a in the presence of XmAb14045 for 48 hours (see FIG. 13). At 48 hours, XmAb14045 with AML patient-derived PBMC induced robust apoptosis (approximately 50% annexin-V positivity), albeit still slightly lower than that induced with normal PBMC. XmAb14045 again induced robust proliferation of both AML patient and healthy donor CD4⁺ and CD8⁺ T cells.

In summary, XmAb14045 induced allogeneic CD123⁺ KG-1a tumor cell killing by both AML patient-derived and normal PBMC. More importantly, XmAb14045 induced autologous leukemic blast cell killing in PBMC from multiple AML patient samples, suggesting that it could also stimulate depletion of leukemic blast cells in AML patients. Additionally, XmAb14045 in the presence of CD123⁺target cells induced both CD4⁺and CD8⁺ T cell activation in AML patient and normal PBMC, indicating that AML patient T cells are fully functional and capable of responding to XmAb14045.

### Example 3

### Antitumor Activity in a Mouse AML Xenograft Model

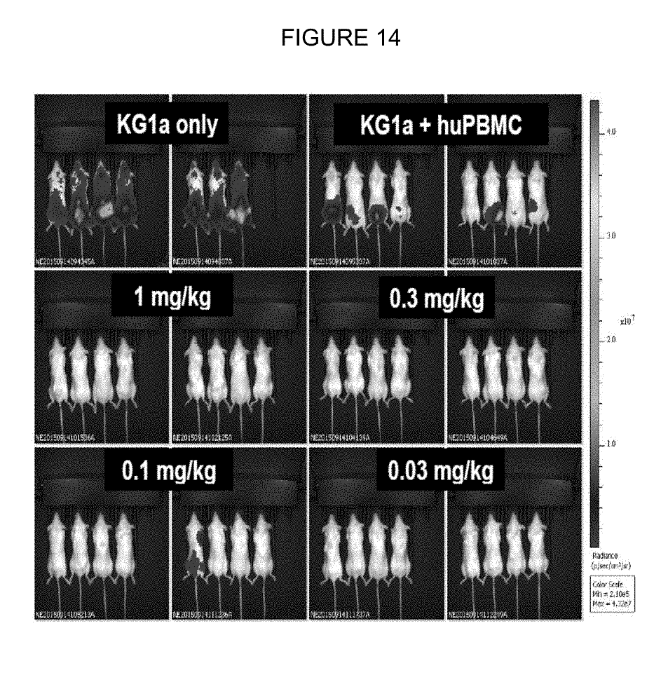

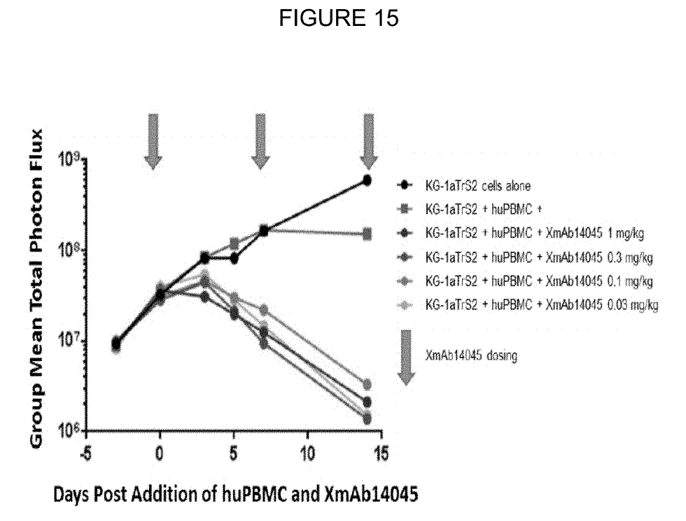

The anti-tumor activity of varying doses of XmAb14045 was examined in NSG mice that were engrafted systemically with KG1aTrS2 cells and normal human PBMCs. KG1aTrS2 cells are derived from the AML cell line KG1a, and have been engineered to express luciferase to allow quantification of tumor burden. Mice received 1×10⁶ KG1aTrS2 cells IV on Day 0. Twenty-two days after injection of KG1aTrS2 cells, mice were engrafted intraperitoneally (IP) with 10×10⁶ PBMC and were treated with 0.03, 0.1, 0.3 or 1.0 mg/kg of XmAb14045 or vehicle once a week for 3 consecutive weeks. Tumor burden was monitored throughout the study by in vivo imaging (FIG. 14). As shown in FIG. 14 and FIG. 15, mice receiving KG1a cells alone or KG1a cells plus PBMC displayed steadily increasing AML burden over time. In contrast, all tested dose levels of XmAb14045 began reducing tumor burden approximately 3 days after the initial dose, ultimately reducing burden by approximately 3 orders of magnitude relative to the KG1a-only control group, and significantly compared to the KG1a-plus-huPBMC group. No significant differences in anti-tumor activity were observed across the XmAb14045 dose range, suggesting that even lower doses would likely still exhibit anti-tumor activity.

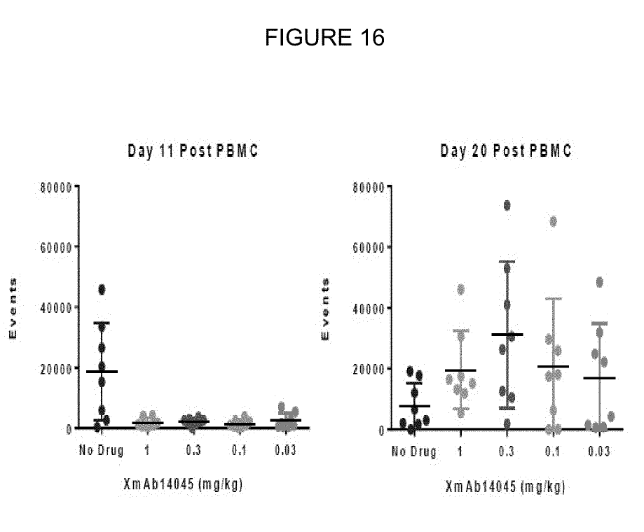

Peripheral blood samples were analyzed by flow cytometry. At Day 11, CD4⁺ and CD8⁺ T cell numbers were decreased in the treated mice compared to control, but by Day 20 this difference was no longer apparent, with a trend toward an increase in T cell counts, suggesting T cell activation and expansion mediated by XmAb14045 (FIG. 16). As another sign of T cell activation, PD1 expression was consistently higher on T cell samples from the XmAb14045-treated groups. However, it is unclear from this study whether the increase in PD1 expression interferes with the activity of XmAb14045.

1. A method for treating a CD123-expressing cancer, e.g., a hematologic cancer, e.g., leukemia, in a human subject, comprising:
administering to the human subject having the CD123-expressing cancer an intravenous dose of between about 1 ng/kg and about 800 ng/kg of a bispecific anti-CD123 x anti-CD3 antibody once every 6-8 days for a time period sufficient to treat the the CD123-expressing cancer.

2. The method of claim 1, wherein the leukemia is selected from the group consisting of acute myeloid leukemia (AML), chronic myeloid leukemia (CML), acute lymphocytic leukemia (ALL), blastic plasmacytoid dendritic cell neoplasm, and hairy cell leukemia (HCL).

3. The method of claim 2, wherein the leukemia is acute myeloid leukemia (AML).

4. The method of claim 3, wherein the acute myeloid leukemia (AML) is blastic plasmacytoid dendritic cell neoplasm (BPDCN).

5. The method of claim 2, wherein the leukemia is acute lymphocytic leukemia, and the acute lymphocytic leukemia is B-cell acute lymphocytic leukemia (B-ALL).

6. The method of claim 1, wherein the intravenous dose is:
between about 2 ng/kg and about 4 ng/kg; or
between about 9 ng/kg and about 11 ng/kg; or
between about 25 ng/kg and about 35 ng/kg; or
between about 70 ng/kg and about 80 ng/kg; or
between about 125 ng/kg and about 175 ng/kg; or
between about 275 ng/kg and about 325 ng/kg; or
between about 475 ng/kg and about 525 ng/kg; or
between about 725 ng/kg and about 775 ng/kg.

7. The method of claim 1, wherein the intravenous dose is administered to the human subject between about 1 hour and about 3 hours.

8. The method of claim 1, wherein the time period sufficient to treat the leukemia is between about 3 weeks and 9 weeks.

9. The method of claim 1, wherein the bispecific anti-CD123 x anti-CD3 antibody comprises: a first monomer comprising SEQ ID NO: 1, a second monomer comprising SEQ ID NO: 2, and a light chain comprising SEQ ID NO: 3.

10. The method of claim 1, further comprising, prior to the administering, assessing the weight of the human subject.

11. A method for treating a CD123-expressing cancer, e.g., a hematologic cancer, e.g., leukemia, in a human subject, comprising:
administering to the human subject having the CD123-expressing cancer an intravenous dose of between about 75 ng/kg and about 750 ng/kg of a bispecific anti-CD123 x anti-CD3 antibody monthly for a time period sufficient to treat the CD123-expressing cancer.

12. A method for treating a CD123-expressing cancer, e.g., a hematologic cancer, e.g., leukemia, in a human subject, comprising:
administering to the human subject having the CD123-expressing cancer an intravenous dose of between about 75 ng/kg and about 750 ng/kg of a bispecific anti-CD123 x anti-CD3 antibody every other week for a time period sufficient to treat the CD123-expressing cancer.

13. The method of claim 11, wherein the leukemia is selected from the group consisting of acute myeloid leukemia (AML), chronic myeloid leukemia (CML), acute lymphocytic leukemia (ALL), blastic plasmacytoid dendritic cell neoplasm, and hairy cell leukemia (HCL).

14. The method of claim 11, wherein the bispecific anti-CD123 x anti-CD3 antibody comprises: a first monomer comprising SEQ ID NO: 1, a second monomer comprising SEQ ID NO: 2, and a light chain comprising SEQ ID NO: 3.

15. The method of claim 11, wherein the time period sufficient to treat the leukemia is between about 4 weeks and 9 weeks.

16. The method of claim 1 further comprising administering to said subject another therapy.

17. The method of claim 16, wherein said another therapy is a chemotherapy.

18. The method of claim 17, wherein said chemotherapy is selected from the group consisting of: a anthracycline (e.g., idarubicin, daunorubicin, doxorubicin (e.g., liposomal doxorubicin)), a anthracenedione derivative (e.g., mitoxantrone), a vinca alkaloid (e.g., vinblastine, vincristine, vindesine, vinorelbine), an alkylating agent (e.g., cyclophosphamide, deacarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, cytarabine, pyrimidine analogs, purine analogs and adenosine deaminase inhibitors (e.g., fludarabine)), an mTOR inhibitor, a proteasome inhibitor (e.g., aclacinomycin A, gliotoxin or bortezomib), an immunomodulator such as thalidomide or a thalidomide derivative (e.g., lenalidomide).

19. The method of claim 16, wherein said another therapy is a therapy that ameliorates side effects.

20. The method of claim 19, wherein said another therapy is selected from the group consisting of: a steroid (e.g., corticosteroid, e.g., methylprednisolone, hydrocortisone), an inhibitor of TNFα, inhibitor of IL-1R, and an inhibitor of IL-6).

21. The method of claim 19, wherein said another therapy is a combination of a corticosterioid (e.g., methylprednisolone, hydrocortisone) and Benadryl and Tylenol, wherein said corticosterioid, Benadryl and Tylenol are administered to said subject prior to the administration of said anti-CD123 x anti-CD3 antibody.


SEQUENCE LISTING (18 sequences)

SEQ ID NO: 1 - protein, 449 aa, Artificial Sequence
  XENP14045 Anti-CD123 x Anti-CD3 Fab-scFv-Fc Heavy Chain 1
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 2 - protein, 485 aa, Artificial Sequence
  XENP14045 Anti-CD123 x Anti-CD3 Fab-scFv-Fc Heavy Chain 2
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 220 aa, Artificial Sequence
  XENP14045 Anti-CD123 x Anti-CD3 Fab-scFv-Fc Light Chain
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 225 aa, Artificial Sequence
  XENP13760 7G3_H0L0_Fab_His Heavy chain
EVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGS

SEQ ID NO: 5 - protein, 220 aa, Artificial Sequence
  XENP13760 7G3_H0L0_Fab_His Light chain
DFVMTQSPSSLTVTAGEKVTMSCKSSQSLLNSGNQKNYLTWYLQKPGQPPKLLIYWASTRESGVPDRFTGSGSGTDFTLTISSVQAEDLAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 225 aa, Artificial Sequence
  XENP13761 7G3_H1L1_Fab_His Heavy chain
QVQLVQSGAEVKKPGASVKVSCKASGYTFTDYYMKWVRQAPGQSLEWMGDIIPSNGATFYNQKFQGRVTMTVDRSTSTAYMELSSLRSEDTAVYYCTRSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGS

SEQ ID NO: 7 - protein, 220 aa, Artificial Sequence
  XENP13761 7G3_H1L1_Fab_His Light chain
DFVMTQSPDSLAVSLGERATINCKSSQSLLNSGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 225 aa, Artificial Sequence
  XENP13961 7G3_H1.107_L1_Fab_His Heavy chain
QVQLQQSGAEVKKPGASVKVSCKASGYTFTDYYMKWVRQAPGQSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGS

SEQ ID NO: 9 - protein, 220 aa, Artificial Sequence; identical to SEQ ID NO: 7
  XENP13961 7G3_H1.107_L1_Fab_His Light chain

SEQ ID NO: 10 - protein, 225 aa, Artificial Sequence
  XENP13963 7G3_H1.109_L1_Fab_His Heavy chain
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGS

SEQ ID NO: 11 - protein, 220 aa, Artificial Sequence; identical to SEQ ID NO: 7
  XENP13963 7G3_ H1.109_L1_Fab_His Light chain

SEQ ID NO: 12 - protein, 225 aa, Artificial Sequence; identical to SEQ ID NO: 8
  XENP13965 7G3_H1.107_L1.57_Fab_His Heavy chain

SEQ ID NO: 13 - protein, 220 aa, Artificial Sequence; identical to SEQ ID NO: 3
  XENP13965 7G3_H1.107_L1.57_Fab_His Light chain

SEQ ID NO: 14 - protein, 225 aa, Artificial Sequence; identical to SEQ ID NO: 10
  XENP13967 7G3_H1.109_L1.57_Fab_His Heavy chain

SEQ ID NO: 15 - protein, 220 aa, Artificial Sequence; identical to SEQ ID NO: 3
  XENP13967 7G3_H1.109_L1.57_Fab_His Light chain

SEQ ID NO: 16 - protein, 449 aa, Artificial Sequence
  XENP13928 Anti-CD123 x Anti-CD3 Fab-scFv-Fc Heavy Chain 1
EVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 17 - protein, 485 aa, Artificial Sequence; identical to SEQ ID NO: 2
  XENP13928 Anti-CD123 x Anti-CD3 Fab-scFv-Fc Heavy Chain 2

SEQ ID NO: 18 - protein, 220 aa, Artificial Sequence; identical to SEQ ID NO: 5
  XENP13928 Anti-CD123 x Anti-CD3 Fab-scFv-Fc Light Chain